SEQUENCE LISTING (75 sequences)

SEQ ID NO: 1 - protein, 8 aa, synthetic construct
GLTFNDYA

SEQ ID NO: 2 - protein, 8 aa, synthetic construct
IRGSGGVT

SEQ ID NO: 3 - protein, 12 aa, synthetic construct
NARPLFGWSEEY

SEQ ID NO: 4 - protein, 119 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSS

SEQ ID NO: 5 - protein, 8 aa, synthetic construct
GFTFSTYN

SEQ ID NO: 6 - protein, 8 aa, synthetic construct
IKWSASTT

SEQ ID NO: 7 - protein, 17 aa, synthetic construct
AGSPHIALGTRTEMYPY

SEQ ID NO: 8 - protein, 124 aa, synthetic construct
DVQLQESGGGLVQPGGSLRLSCATSGFTFSTYNMGWFRQAPGKEREFVGSIKWSASTTYYADSVKGRFTISRDNAKNTVYLQMNSLKPEDTAIYYCAGSPHIALGTRTEMYPYWGQGTQVTVSS

SEQ ID NO: 9 - protein, 141 aa, synthetic construct
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 137 aa, synthetic construct
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 11 - protein, 137 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 12 - protein, 133 aa, synthetic construct
DIQNPDPAVYQLRDDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 13 - protein, 135 aa, synthetic construct
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLW

SEQ ID NO: 14 - protein, 135 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLW

SEQ ID NO: 15 - protein, 131 aa, synthetic construct
DIQNPDPAVYQLRDDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLW

SEQ ID NO: 16 - protein, 176 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDF

SEQ ID NO: 17 - protein, 172 aa, synthetic construct
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - protein, 172 aa, synthetic construct
DLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 19 - protein, 166 aa, synthetic construct
DLKNVFPPEVAVFEPSAEIATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 20 - protein, 166 aa, synthetic construct
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 21 - protein, 166 aa, synthetic construct
DLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 22 - protein, 160 aa, synthetic construct
DLKNVFPPEVAVFEPSAEIATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 23 - protein, 37 aa, synthetic construct
RRDQRLPPDAHKPPGGGSFRTPIQEEQADAHSTLAKI

SEQ ID NO: 24 - protein, 15 aa, synthetic construct
GGGGSGGGGSGGGGS

SEQ ID NO: 25 - protein, 15 aa, synthetic construct
EAAAKEAAAKEAAAK

SEQ ID NO: 26 - protein, 27 aa, synthetic construct
RRKRSGSGATNFSLLKQAGDVEENPGP

SEQ ID NO: 27 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 237 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 29 - protein, 26 aa, synthetic construct
SGGGSGGGGSGGGGSGGGGSGGGSLQ

SEQ ID NO: 30 - protein, 377 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 31 - protein, 8 aa, synthetic construct
GVSFSPYG

SEQ ID NO: 32 - protein, 7 aa, synthetic construct
IYRSGST

SEQ ID NO: 33 - protein, 7 aa, synthetic construct
QILGTSY

SEQ ID NO: 34 - protein, 113 aa, synthetic construct
DVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSS

SEQ ID NO: 35 - DNA, 400 bp, synthetic construct
  source 1..400: mol_type = other DNA
aatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcccggtcgccacc

SEQ ID NO: 36 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 37 - protein, 18 aa, synthetic construct
MDWTWILFLVAAATRVHS

SEQ ID NO: 38 - protein, 22 aa, synthetic construct
MLLLVTSLLLCELPHPAFLLIP

SEQ ID NO: 39 - protein, 323 aa, synthetic construct
DVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 40 - protein, 174 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 41 - protein, 8 aa, synthetic construct
GFTFNYYH

SEQ ID NO: 42 - protein, 8 aa, synthetic construct
IRSNADDT

SEQ ID NO: 43 - protein, 14 aa, synthetic construct
ARISAWRRPKEYDY

SEQ ID NO: 44 - protein, 121 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSS

SEQ ID NO: 45 - protein, 10 aa, synthetic construct
GFTLDYYAIG

SEQ ID NO: 46 - protein, 10 aa, synthetic construct
CVSSSDGSTY

SEQ ID NO: 47 - protein, 18 aa, synthetic construct
DQYSSTWTIRLTRCHFGS

SEQ ID NO: 48 - protein, 127 aa, synthetic construct
DVQLQESGGGLVQPGGSLRLSCAASGFTLDYYAIGWFRQAPGKEREGVSCVSSSDGSTYYADSVKGRFTISRDNAKNTVYLQMNSLKPEDTAVYYCAADQYSSTWTIRLTRCHFGSWGQGTQVTVSS

SEQ ID NO: 49 - protein, 292 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 50 - protein, 187 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWGGGGSGGGGSGGGGSRRDQRLPPDAHKPPGGGSFRTPIQEEQADAHSTLAKI

SEQ ID NO: 51 - protein, 297 aa, synthetic construct
DVQLQESGGGLVQPGGSLRLSCATSGFTFSTYNMGWFRQAPGKEREFVGSIKWSASTTYYADSVKGRFTISRDNAKNTVYLQMNSLKPEDTAIYYCAGSPHIALGTRTEMYPYWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 52 - protein, 187 aa, synthetic construct; identical to SEQ ID NO: 50

SEQ ID NO: 53 - protein, 331 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 54 - protein, 174 aa, synthetic construct; identical to SEQ ID NO: 40

SEQ ID NO: 55 - protein, 329 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 56 - protein, 295 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 57 - protein, 331 aa, synthetic construct; identical to SEQ ID NO: 53

SEQ ID NO: 58 - protein, 293 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 59 - protein, 323 aa, synthetic construct; identical to SEQ ID NO: 39

SEQ ID NO: 60 - protein, 293 aa, synthetic construct; identical to SEQ ID NO: 58

SEQ ID NO: 61 - protein, 329 aa, synthetic construct; identical to SEQ ID NO: 55

SEQ ID NO: 62 - protein, 287 aa, synthetic construct
DVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 63 - protein, 300 aa, synthetic construct
DVQLQESGGGLVQPGGSLRLSCAASGFTLDYYAIGWFRQAPGKEREGVSCVSSSDGSTYYADSVKGRFTISRDNAKNTVYLQMNSLKPEDTAVYYCAADQYSSTWTIRLTRCHFGSWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 64 - protein, 187 aa, synthetic construct; identical to SEQ ID NO: 50

SEQ ID NO: 65 - protein, 329 aa, synthetic construct; identical to SEQ ID NO: 55

SEQ ID NO: 66 - protein, 301 aa, synthetic construct
DVQLQESGGGLVQPGGSLRLSCAASGFTLDYYAIGWFRQAPGKEREGVSCVSSSDGSTYYADSVKGRFTISRDNAKNTVYLQMNSLKPEDTAVYYCAADQYSSTWTIRLTRCHFGSWGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 67 - protein, 337 aa, synthetic construct
DVQLQESGGGLVQPGGSLRLSCAASGFTLDYYAIGWFRQAPGKEREGVSCVSSSDGSTYYADSVKGRFTISRDNAKNTVYLQMNSLKPEDTAVYYCAADQYSSTWTIRLTRCHFGSWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 68 - protein, 293 aa, synthetic construct; identical to SEQ ID NO: 58

SEQ ID NO: 69 - protein, 347 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 70 - protein, 352 aa, synthetic construct
DVQLQESGGGLVQPGGSLRLSCATSGFTFSTYNMGWFRQAPGKEREFVGSIKWSASTTYYADSVKGRFTISRDNAKNTVYLQMNSLKPEDTAIYYCAGSPHIALGTRTEMYPYWGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 71 - protein, 349 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 72 - protein, 488 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSGGGGSGGGGSGGGGSGGGGSDVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 73 - protein, 488 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSGGGGSGGGGSGGGGSGGGGSDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 74 - protein, 9 aa, synthetic construct
LSVMGLRIL

SEQ ID NO: 75 - protein, 9 aa, synthetic construct
LLVIVLRIL

(19) United States
(12) Patent Application Publication
(10) Pub. No.: US 2025/0312452 A1
(43) Pub. Date: Oct. 9, 2025

(54) ANTIBODY TARGETING CLAUDIN18.2 AND ITS APPLICATIONS

(71) Applicant: BRISTAR IMMUNOTECH LIMITED, Beijing (CN)

(72) Inventors: Yang LI, Beijing (CN); Lei LEI, Beijing (CN); Dongmei YANG, Beijing (CN); Zhe WAN, Beijing (CN)

(21) Appl. No.: 19/088,847

(22) Filed: Mar. 24, 2025

(30) Foreign Application Priority Data
Apr. 9, 2024 (CN) 202410424073.7

(51) Int. Cl. A61K 40/42; A61K 38/20; A61K 40/11; A61K 40/31; C07K 14/54; C07K 16/28; C07K 16/30

(52) U.S. Cl. CPC A61K 40/4202; A61K 38/2086; A61K 40/11; A61K 40/31; A61K 40/421; A61K 40/4219; A61K 40/4255; C07K 14/5443; C07K 16/28; C07K 16/2803; C07K 16/2866; C07K 16/30; C07K 2317/569; C07K 2317/92

(57) ABSTRACT

The present invention belongs to the field of biomedicine. Specifically, the present invention provides an antibody targeting Claudin18.2 and its applications. More specifically, the present invention provides an antibody targeting Claudin18.2, a STAR derived from the antibody, therapeutic immune cells containing the STAR, and their applications in the treatment of diseases.

### CROSS-REFERENCE TO RELATED APPLICATIONS

The present application claims the priority benefit of Chinese Patent Application No. 202410424073.7, filed Apr. 9, 2024, which is herein incorporated by reference in its entirety.

### REFERENCE TO AN ELECTRONIC SEQUENCE LISTING

The contents of the electronic sequence listing (8575-0083_Sequence listing.xml; Size: 78246 bytes; and Date of Creation: 03/13/2025) is herein incorporated by reference in its entirety.

### TECHNICAL FIELD

The present invention belongs to the field of biomedicine. Specifically, the present invention provides an antibody targeting Claudin18.2 and its applications. More particularly, the present invention provides an antibody targeting Claudin18.2, a STAR derived from the antibody, therapeutic immune cells comprising the STAR, and their use in the treatment of diseases.

### BACKGROUND

Claudin proteins are key components of the tight junction complex, playing a significant role in human life activities. On one hand, tight junctions serve the function of a “barrier”, selecting substances based on size and charge, thereby regulating paracellular transport. For example, the blood-brain barrier formed by cerebrovascular endothelial cells prevents the mixing of blood and brain extracellular fluid, etc. On the other hand, tight junctions also serve the function of a “fence,” maintaining cell polarity by controlling the free diffusion of lipids and proteins between the apical and basolateral membrane domains.

Claudin18.2 is a member of the Claudin protein family, characterized by its highly tissue-specific expression, normally found only in differentiated epithelial cells of the gastric mucosa. Research has revealed that Claudin18.2 is highly expressed in various tumors, including gastric cancer, pancreatic cancer, esophageal adenocarcinoma, and colorectal adenocarcinoma, etc. This unique expression pattern-limited in normal tissues but highly specific in tumors-makes Claudin18.2 an ideal target for the development of immunotherapies for solid tumors.

Based on the high similarity between the extracellular domain of the TCR and the Fab region of antibodies, replacing the variable region sequences of the TCR with antibody variable region sequences (such as scFv) results in the creation of Synthetic T-Cell Receptor and Antibody Receptor (STAR). STAR combines the advantages of both TCR and CAR, possessing the specificity of antibodies and the superior signaling capabilities of natural TCRs. The STAR can mediate complete T cell activation, with significant improvements in safety and efficacy, making it a promising new type of cellular immunotherapy.

Providing new and more effective antibodies targeting Claudin18.2 and combining them with STAR technology is expected to provide a more effective approach for solid tumors.

### SUMMARY OF THE INVENTION

The present invention relates to a single-domain antibody that specifically binds to Claudin18.2 and its application in Synthetic T-cell Receptor and Antigen Receptors (STAR) and Chimeric Antigen Receptors (CAR). The single-domain antibody contains specific CDR1, CDR2, and CDR3 sequences, enabling it to bind to Claudin18.2 with high efficiency and specificity. Based on this single-domain antibody, the present invention further develops a variety of STAR and CAR structures targeting Claudin18.2, which can be used to treat a variety of Claudin18.2-related diseases.

The core content of the present invention includes the following aspects: Development of Single-Domain Antibodies: The present invention provides a single-domain antibody that specifically binds to Claudin18.2, comprising specific CDR1, CDR2 and CDR3. For example, the CDR1, CDR2 and CDR3 sequences are selected from CDR1, CDR2 and CDR3 in SEQ ID NO:4 or 8, wherein the antibody is a murine-derived antibody or a humanized antibody, with high specificity and affinity.

Synthetic T-cell Receptor and Antigen Receptor (STAR): The present invention provides a STAR targeting Claudin18.2, the antigen-binding region of the STAR contains the single-domain antibody.

The STAR structure can include one or more antigen-binding regions and can be combined with antibodies or antigen-binding fragments that specifically bind to other antigens (e.g., MSLN, LILRB4 or CCR8) to form dual-targeting structures.

Chimeric Antigen Receptor (CAR): The present invention provides a CAR targeting Claudin18.2, an extracellular antigen-binding region of the CAR contains the single-domain antibody. The CAR structure can also include antigen-binding regions that specifically bind to other antigens to achieve multi-targeting therapy.

Nucleic Acid Molecules and Expression Vectors: The present invention provides nucleic acid molecules encoding the single-domain antibody, STAR, and CAR, as well as expression vectors containing these nucleic acid molecules. These expression vectors can be used to express the corresponding protein structures in host cells.

Host Cells and Therapeutic Immune Cells: The present invention provides cells expressing the single-domain antibody, STAR, or CAR obtained by transforming host cells. These cells can be further used to prepare therapeutic immune cells, such as T cells or NK cells.

Pharmaceutical Compositions and Therapeutic Uses: The present invention provides pharmaceutical compositions containing the single-domain antibody, STAR, CAR, or therapeutic immune cells for treating various Claudin18.2-related diseases, particularly hematologic tumors and solid tumors.

The single-domain antibody, STAR, and CAR structures provided by the present invention exhibit high specificity and affinity, and can effectively target Claudin18.2. The combination with other antigen-binding regions can enhance therapeutic efficacy. Through the development of nucleic acid molecules and expression vectors, the present invention provides a foundation for large-scale production and application. Additionally, the present invention covers the use of these structures in pharmaceutical compositions, offering new strategies for treating various diseases.

In a first aspect of the present invention, provided is a single-domain antibody specifically binding to Claudin18.2, comprising CDR1, CDR2, and CDR3 sequences selected from CDR1, CDR2 and CDR3 in SEQ ID NO:4 or 8.

In some embodiments, the single-domain antibody comprises the CDR1 has an amino acid sequence set forth in SEQ ID NO: 5, wherein the CDR2 has an amino acid sequence set forth in SEQ ID NO: 6, wherein the CDR3 has an amino acid sequence set forth in SEQ ID NO: 7.

In some embodiments, the single-domain antibody comprises the CDR1 has an amino acid sequence set forth in SEQ ID NO:1, wherein the CDR2 has an amino acid sequence set forth in SEQ ID NO: 2, wherein the CDR3 has an amino acid sequence set forth in SEQ ID NO: 3.

In some embodiments, the single-domain antibody comprises the amino acid sequence has at least 80%, preferably at least 90%, more preferably at least 95%, and even more preferably at least 99% identity to SEQ ID NO: 8, preferably, wherein the amino acid sequence shown in SEQ ID NO: 4 or 8.

In some embodiments, the single-domain antibody is a murine-derived antibody or a humanized antibody.

In second aspect of the present invention, provided is a synthetic T-cell receptor and antigen receptor (STAR) targeting Claudin18.2, an antigen-binding region of the STAR comprises the single-domain antibody specifically binding to Claudin18.2.

In some embodiments, the STAR comprises a first peptide chain and a second peptide chain:
i) the first peptide chain comprises a first target-binding region and a first constant region, and the second peptide chain comprises a second target-binding region and a second constant region; or,
ii) the first peptide chain comprises a first constant region, the second peptide chain comprises a second constant region, and the first peptide chain or the second peptide chain comprises a first target-binding region;
wherein the first target-binding region and/or the second target-binding region comprises one or more antigen-binding regions, and the multiple antigen-binding regions are the same or different; the antigen-binding region in the first target-binding region and/or the second target-binding region comprises the Claudin18.2 single-domain antibody.

In some embodiments, the STAR comprises the antigen-binding region in the first target-binding region and/or the second target-binding region further comprises an antibody or antigen-binding fragment specifically binding to MSLN, LILRB4 or CCR8;
preferably, the antibody or antigen-binding fragment is selected from scFv, nanobody, or single-domain antibody.

In some embodiments, the STAR comprises any one of the following groups:
a) the first peptide chain comprises a first constant region; the second peptide chain sequentially comprises, from the N-terminus to the C-terminus, at least one Claudin18.2 single-domain antibody, and a second constant region;
b) the first peptide chain sequentially comprises, from the N-terminus to the C-terminus, the Claudin18.2 single-domain antibody, and a first constant region; the second peptide chain sequentially comprises, from the N-terminus to the C-terminus, an antibody or antigen-binding fragment specifically binding to MSLN, LILRB4 or CCR8, and a second constant region;
c) the first peptide chain comprises a first constant region; the second peptide chain sequentially comprises, from the N-terminus to the C-terminus, at least one Claudin18.2 single-domain antibody, an antibody or an antigen-binding fragment specifically binding to MSLN, LILRB4 or CCR8, and a second constant region;

In the each of the above group a)-c), the first constant region of the first peptide chain is a TCRα chain constant region or a TCRβ chain constant region, and the second constant region of the second peptide chain is a TCRβ chain constant region or a TCRα chain constant region; the constant regions of the first peptide chain and the second peptide chain are not simultaneously TCRα chain constant regions, nor are they simultaneously TCRβ chain constant regions.

In some embodiments, the antibody or antigen-binding fragment specifically binding to MSLN comprises CDR1 as shown in SEQ ID NO:31, CDR2 as shown in SEQ ID NO:32, and CDR3 as shown in SEQ ID NO:33; preferably, the antibody or antigen-binding fragment specifically binding to MSLN comprises a single-domain antibody with the amino acid sequence shown in SEQ ID NO:34; the antibody or antigen-binding fragment specifically binding to CCR8 comprises CDR1 as shown in SEQ ID NO:41, CDR2 as shown in SEQ ID NO:42, and CDR3 as shown in SEQ ID NO:43; preferably, the antibody or antigen-binding fragment specifically binding to CCR8 comprises a single-domain antibody with the amino acid sequence shown in SEQ ID NO:44;
the antibody or antigen-binding fragment specifically binding to LILRB4 comprises CDR1 as shown in SEQ ID NO:45, CDR2 as shown in SEQ ID NO:46, and CDR3 as shown in SEQ ID NO:47; preferably, the antibody or antigen-binding fragment specifically binding to LILRB4 comprises a single-domain antibody with the amino acid sequence shown in SEQ ID NO:48.

In some embodiments, wherein the first constant region is a TCRα chain constant region or a TCRβ chain constant region, preferably a modified TCRα chain constant region or TCRβ chain constant region; wherein the second constant region is a TCRα chain constant region or a TCRβ chain constant region, preferably a modified TCRα chain constant region or TCRβ chain constant region; preferably, the TCRα chain constant region is selected from the constant region of a wild-type human or wild-type mouse TCRα chain; preferably, the TCRβ chain constant region is selected from the constant region of a wild-type human or wild-type mouse TCRβ chain.

In some embodiments, wherein the modified TCRα chain constant region is derived from a mouse TCRα chain constant region, the amino acids at positions 6, 13, 15-18, 48, 112, 114, 115, 122, 136, and 137 comprise one or more modifications, as compared to the wild-type mouse TCRα chain constant region, wherein the modifications are amino acid substitutions or deletions; wherein the modified TCRβ chain constant region is derived from a mouse TCRβ chain constant region, the amino acids at positions 3, 6, 9, 11, 12, 17, 21-25, 56, 150, 162-172, 168, or 170 comprise one or more modifications, as compared to the wild-type mouse TCRβ chain constant region, wherein the modifications are amino acid substitutions or deletions; or In some embodiments, wherein the modified TCRα chain constant region is derived from a human TCRα chain constant region, the amino acids at positions 47, 90-93, 115, and 118 comprise one or more modifications, as compared to the wild-type human TCRα chain constant region, wherein the modifications are amino acid substitutions or deletions; and/or wherein the modified TCRβ chain constant region is derived from a human TCRβ chain constant region, the amino acids at positions 17, 21, 56, 132, 135, and 138 comprise one or more modifications, as compared to the wild-type human TCRβ chain constant region, wherein the modifications are amino acid substitutions or deletions.

In some embodiments, wherein the modified TCRα chain constant region is derived from a wild-type mouse TCRα chain constant region, as compared to the wild-type mouse TCRα chain constant region, and contains amino acid mutations selected from the following group or combinations thereof:
i) the amino acid threonine (T) at position 48 is mutated to Cysteine (C),
ii) the amino acid serine (S) at position 112 is mutated to leucine (L), the amino acid methionine (M) at position 114 is mutated to isoleucine (I), and/or the amino acid Glycine (G) at position 115 is mutated to valine (V),
iii) the amino acid E at position 6 is substituted by D, and the amino acid K at position 13 is substituted by R, the amino acids at positions 15-18 are deleted;
iv) the amino acid K at position 122 is substituted by R;
v) and lacks the endodomain of the constant region, for example, the amino acids at positions 136-137 are deleted;
and/or, wherein the modified TCRβ chain constant region is derived from a wild-type mouse TCRβ chain constant region, as compared to the wild-type mouse TCRβ chain constant region, and contains amino acid mutations selected from the following group or combinations thereof:
i) the amino acid serine (S) at position 56 is mutated to cysteine (C),
ii) the amino acids at positions 150, 168, and 170 are all substituted by R,
iii) the amino acid R at position 3 is substituted by K, the amino acid T at position 6 is substituted by F, K at position 9 is substituted by E, S at position 11 is substituted by A, and L at position 12 is substituted by V, and/or amino acids at positions 17, 21-25 are deleted,
iv) and lacks the endodomain of the constant region, for example, the amino acids at positions 167-172 are deleted.

In some embodiments, wherein the modified TCRα chain constant region is derived from a wild-type human TCRα chain constant region, as compared to the wild-type human TCRα chain constant region, and contains amino acid mutations selected from the following group or combinations thereof:
i) the amino acid threonine (T) at position 47 is mutated to Cysteine (C);
ii) the amino acid P at position 90 is mutated to S, the amino acid E at position 91 is mutated to D, the amino acid S at position 92 is mutated to V, the amino acid S at position 93 is mutated to P;
iii) the amino acid S at position 115 is mutated to L, the amino acid G at position 118 is mutated to V;
and/or, wherein the modified TCRβ chain constant region is derived from a wild-type human TCRβ chain constant region, as compared to the wild-type human TCRβ chain constant region, and contains amino acid mutations selected from the following group or combinations thereof:
i) the amino acid serine (S) at position 56 is mutated to cysteine (C),
ii) the amino acid E at position 17 is substituted by K, the amino acid S at position 21 is substituted by A, F at position 132 is substituted by I, E at position 135 is substituted by A, and/or Q at position 138 is substituted by H.

In some embodiments, wherein the TCRα chain constant region comprises an amino acid sequence shown in one of SEQ ID NOs:9-15, and/or the TCRβ chain constant region comprises an amino acid sequence shown in one of SEQ ID NOs:16-22.

In some embodiments, wherein the first peptide chain and/or the second peptide chain has at least one exogenous intracellular functional domain linked to its C-terminus to, such as the endodomain of a co-stimulatory molecule, preferably an endodomain of OX40, more preferably, the endodomain of OX40 comprises the amino acid sequence of SEQ ID NO:23.

In some embodiments, wherein the exogenous intracellular functional domain is linked directly or via a linker to the C-terminus of the constant region of the first peptide chain and/or the second peptide chain, preferably, the exogenous intracellular functional domain is linked to the C-terminus of the constant region of the first peptide chain and/or the second peptide chain whose endodomain is deleted, through a linker, preferably, the linker is a (G4S)n linker or an (EAAAK)n linker, where n represents an integer from 1 to 10, preferably, n is 3 or 4.

In some embodiments, the STAR is co-expressed with a membrane-bound IL-15 protein (mbIL-15).

In some embodiments, wherein:
a) the STAR comprises a first peptide chain shown in SEQ ID NO:49 and a second peptide chain shown in SEQ ID NO:50
b) the STAR comprises a first peptide chain shown in SEQ ID NO:51 and a second peptide chain shown in SEQ ID NO:52;
c) the STAR comprises a first peptide chain shown in SEQ ID NO:61 and a second peptide chain shown in SEQ ID NO:62;
d) the STAR comprises a first peptide chain shown in SEQ ID NO:59 and a second peptide chain shown in SEQ ID NO:60;
e) the STAR comprises a first peptide chain shown in SEQ ID NO:55 and a second peptide chain shown in SEQ ID NO:56;
f) the STAR comprises a first peptide chain shown in SEQ ID NO:57 and a second peptide chain shown in SEQ ID NO:58;
g) the STAR comprises a first peptide chain shown in SEQ ID NO:65 and a second peptide chain shown in SEQ ID NO:66; or
h) the STAR comprises a first peptide chain shown in SEQ ID NO:67 and a second peptide chain shown in SEQ ID NO:68.

In some embodiments, wherein:
i) the amino acid sequence of IL-15 is shown in SEQ ID NO: 27;
ii) the amino acid sequence of the extracellular domain of IL-15Rα is shown in SEQ ID NO: 28;
iii) the amino acid sequence of the linker connecting the extracellular domain of IL-15Rα to IL-15 is shown in SEQ ID NO: 24-25, 29; and/or
iv) the amino acid sequence of mbIL-15 is shown in SEQ ID NO: 30.

In third aspect of the present invention, provided is a chimeric antigen receptor (CAR) targeting Claudin18.2, comprising an extracellular antigen-binding region, wherein the extracellular antigen-binding region comprises the Claudin18.2 single-domain antibody, and the CAR sequentially comprises, from the N-terminus to the C-terminus, an extracellular antigen-binding region, a hinge region, a transmembrane domain, a co-stimulatory domain, and an intracellular signaling-transductiondomain.

In some embodiments, wherein the extracellular antigen-binding region further comprises an antigen-binding region specifically binding to another antigen, preferably, the antigen-binding region specifically binding to the other antigen comprises a single-chain antibody (scFv) or single-domain antibody specifically binding to the other antigen.

In some embodiments, wherein the other antigen is CCR8, preferably, the antigen-binding region specifically binding to CCR8 comprises CDR1 as shown in SEQ ID NO:41, CDR2 as shown in SEQ ID NO:42, and CDR3 as shown in SEQ ID NO:43, preferably, the antigen-binding region specifically binding to CCR8 comprises the amino acid sequence shown in SEQ ID NO:44;
wherein the other antigen is MSLN, preferably, the antigen-binding region specifically binding to MSLN comprises CDR1 as shown in SEQ ID NO:31, CDR2 as shown in SEQ ID NO:32, and CDR3 as shown in SEQ ID NO:33, preferably, the antigen-binding region specifically binding to MSLN comprises the amino acid sequence shown in SEQ ID NO:34; or
wherein the other antigen is LILRB4, preferably, the antigen-binding region specifically binding to LILRB4 comprises CDR1 as shown in SEQ ID NO:45, CDR2 as shown in SEQ ID NO:46, and CDR3 as shown in SEQ ID NO:47, preferably, the antigen-binding region specifically binding to LILRB4 comprises the amino acid sequence shown in SEQ ID NO:48.

In some embodiments, wherein the CAR comprises an amino acid sequence shown in any one of SEQ ID NOs:69-73.

In fourth aspect of the present invention, provided is an isolated nucleic acid molecule encoding the aforementioned single-domain antibody, or the aforementioned STAR, or the aforementioned CAR.

In fifth aspect of the present invention, provided is an expression vector comprising the aforementioned nucleic acid molecule, wherein the nucleic acid molecule is operably linked to an expression regulatory element such as a promoter.

In some embodiments, wherein the expression vector comprises:
1) a coding nucleotide sequence of a fusion polypeptide of the first peptide chain of the STAR and the second peptide chain of the STAR connected by a self-cleaving peptide; or
2) a coding nucleotide sequence of a fusion polypeptide of the first peptide chain of the STAR, the second peptide chain of the STAR, and mbIL-15 connected by a self-cleaving peptide.

In some embodiments, wherein the self-cleaving peptide is a 2A peptide, preferably, the self-cleaving peptide is a Furin-2A peptide, such as the Furin-P2A peptide shown in SEQ ID NO:26.

In sixth aspect of the present invention, provided is a host cell obtained by transforming a cell with the aforementioned nucleic acid molecule or the aforementioned expression vector.

In seventh aspect of the present invention, provided is an isolated therapeutic immune cell comprising the aforementioned STAR or the aforementioned CAR.

In some embodiments, wherein the immune cell is a T cell or natural killer (NK) cell, preferably a T cell.

In eighth aspect of the present invention, provided is a method for preparing the aforementioned therapeutic immune cell, comprising:
Step 1) providing a starting immune cell;
Step 2) introducing the aforementioned expression vector into the starting immune cell; and
Step 3) harvesting the immune cell obtained in Step 2.

In ninth aspect of the present invention, provided is a method for producing a single-domain antibody specifically binding to Claudin18.2, comprising:
(i) culturing the aforementioned host cell under conditions suitable for expression of the nucleic acid molecule or expression vector, and
(ii) isolating and purifying the single-domain antibody specifically binding to Claudin18.2 expressed by the host cell.

In tenth aspect of the present invention, provided is a pharmaceutical composition comprising the aforementioned single-domain antibody, the aforementioned STAR, the aforementioned CAR, the aforementioned therapeutic immune cell, and/or the aforementioned expression vector, and a pharmaceutically acceptable carrier, preferably for treating a disease in a subject.

In eleventh aspect of the present invention, provided is the use of the aforementioned single-domain antibody, the aforementioned STAR, the aforementioned CAR, the aforementioned therapeutic immune cell, and/or the aforementioned expression vector and/or the aforementioned pharmaceutical composition in the preparation of a medicament for treating a disease in a subject.

In some embodiments, wherein the disease is a Claudin18.2-related disease, such as a Claudin18.2-related autoimmune disease, hematologic tumor, or solid tumor;
preferably, wherein the autoimmune disease is selected from the following group: systemic lupus erythematosus (SLE), polymyositis and dermatomyositis, systemic scleroderma, Sjögren's syndrome, autoimmune hemolytic anemia, or rheumatoid arthritis;

Preferably, wherein the tumor is selected from the following group: lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, digestive tract cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma; More preferably, the tumor is selected from the following group: rectal cancer, breast cancer, gastric cancer, metastatic brain cancer, metastatic liver cancer, lung cancer, digestive tract cancer, mesothelioma, pancreatic cancer, ovarian cancer, or B-cell malignancies.

In twelfth aspect of the present invention, provided is a method for treating a disease in a subject. The method comprises administering to the subject a therapeutically effective amount of the single-domain antibody as described in any one of the preceding items, the STAR as described in any one of the preceding items, the CAR as described above, the therapeutic immune cells as described above, and/or the expression vector as described in any one of the preceding items and/or the pharmaceutical composition as described above. Preferably, the disease is a Claudin18.2-related disease, such as a Claudin18.2-related autoimmune disease, hematologic tumor, or solid tumor;
preferably, wherein the autoimmune disease is selected from the following group: systemic lupus erythematosus (SLE), polymyositis and dermatomyositis, systemic scleroderma, Sjögren's syndrome, autoimmune hemolytic anemia, or rheumatoid arthritis;

Preferably, wherein the tumor is selected from the following group: lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, digestive tract cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma; More preferably, the tumor is selected from the following group: rectal cancer, breast cancer, gastric cancer, metastatic brain cancer, metastatic liver cancer, lung cancer, digestive tract cancer, mesothelioma, pancreatic cancer, ovarian cancer, or B-cell malignancies.

### BRIEF DESCRIPTION OF THE DRAWINGS

FIG. 1: EC50 affinity detection results of candidate antibodies using SNU1-hClaudin18.2 cells.

FIG. 2: EC50 affinity detection results of candidate antibodies using SNU1-hClaudin18.1 cells.

FIG. 3: Specificity detection results of Claudin18.2 nanobody MPA.

FIG. 4: Schematic diagram of the Claudin18.2 STAR nucleic acid construct.

FIG. 5A: Comparison of in vitro killing efficiency of Claudin18.2 STAR against SNU1-hClaudin18.2 target cells.

FIG. 5B: Comparison of in vitro killing efficiency of Claudin18.2 STAR against KATOIII target cells.

FIG. 6: Specific killing test results of Claudin18.2 STAR-T cells against tumor cells expressing Claudin18.1 targets.

FIG. 7A: Comparison of cytokine (IL2) secretion promotion by Claudin18.2 STAR-T cells.

FIG. 7B: Comparison of cytokine (IFN-γ) secretion promotion by Claudin18.2 STAR-T cells.

FIG. 7C: Comparison of cytokine (TNF-α) secretion promotion by Claudin18.2 STAR-T cells.

FIG. 8: Comparison of tumor suppression effects of Claudin18.2 STAR-T cells in mice.

FIG. 9A: Detection results of tumor suppression effects (Total Flux [p/s]) of Claudin18.2 STAR-T cells in mice.

FIG. 9B: Detection results of tumor suppression effects (Tumor Volume) of Claudin18.2 STAR-T cells in mice.

FIG. 9C: Detection results of mice weight of Claudin18.2 STAR-T cells in mice.

FIG. 10A: In vitro killing detection results of SNU1-hClaudin18.2 target cells using dual-target MSLN-Claudin18.2 STAR.

FIG. 10B: In vitro killing detection results of SKOV3 target cells using dual-target MSLN-Claudin18.2 STAR.

FIG. 10C: In vitro killing detection results of KATOIII target cells using dual-target MSLN-Claudin18.2 STAR.

FIG. 11: Tumor growth inhibition by 3E6 dose Claudin18.2 STAR-T cells in vivo.

FIG. 12: Fluorescence test results of tumor killing by 3E6 dose Claudin18.2 STAR-T cells in vivo.

FIG. 13: Body weight changes in mice treated with 3E6 dose Claudin18.2 STAR-T cells in vivo.

FIG. 14: Tumor growth inhibition by 6E6 dose Claudin18.2 STAR-T cells in vivo.

FIG. 15: Fluorescence test results of tumor killing by 6E6 dose Claudin18.2 STAR-T cells in vivo.

FIG. 16: Body weight changes in mice treated with 6E6 dose Claudin18.2 STAR-T cells in vivo.

FIG. 17: In vitro killing activity of CCR8-Claudin18.2 STAR co-expressing mbIL-15 against Claudin18.2-positive 293T cells.

FIG. 18: In vitro killing activity of CCR8-Claudin18.2 STAR co-expressing mbIL-15 against CCR8-positive cells.

FIG. 19A: For 293T-hClaudin18.2 target cells, cytokine secretion detection results stimulated by CCR8-Claudin18.2 STAR co-expressing mbIL-15.

FIG. 19B: For 293T-hCCR8 target cells, cytokine secretion detection results stimulated by CCR8-Claudin18.2 STAR co-expressing mbIL-15.

FIG. 20: Tumor growth inhibition by 3E6 dose CCR8-Claudin18.2 dual-target STAR-T cells in vivo.

FIG. 21: Body weight changes in mice treated with 3E6 dose CCR8-Claudin18.2 dual-target STAR-T cells in vivo.

FIG. 22: Fluorescence test results of tumor killing by 3E6 dose CCR8-Claudin18.2 dual-target STAR-T cells in vivo.

FIG. 23: Tumor volume changes in mice treated with 3E6 dose CCR8-Claudin18.2 dual-target STAR-T cells in vivo.

FIG. 24: Tumor volume inhibition rate in mice treated with 3E6 dose CCR8-Claudin18.2 dual-target STAR-T cells in vivo.

FIG. 25: In vitro killing detection results of Claudin18.2-positive cells by LILRB4-Claudin18.2 dual-target STAR-T cells.

FIG. 26: In vitro killing detection results of LILRB4-positive cells by LILRB4-Claudin18.2 dual-target STAR-T cells.

FIG. 27A: For 293T-hClaudin18.2 target cells, cytokine secretion detection results of LILRB4-Claudin18.2 STAR cells co-expressing mbIL-15.

FIG. 27B: For 293T-hLILRB4 target cells, cytokine secretion detection results of LILRB4-Claudin18.2 STAR cells co-expressing mbIL-15.

FIG. 28: Schematic diagram of Claudin18.2 and dual-target Claudin18.2-CCR8 CAR-T structures.

FIG. 29A: For 293T cells, in vitro killing test results of Claudin18.2-CCR8 CAR-T against tumor cells.

FIG. 29B: For 293T target cells overexpressing hClaudin18.2, in vitro killing test results of Claudin18.2-CCR8 CAR-T against tumor cells.

FIG. 29C: For 293T target cells overexpressing hCCR8, in vitro killing test results of Claudin18.2-CCR8 CAR-T against tumor cells.

### DETAILED DESCRIPTION

### Definitions

Unless otherwise indicated or defined, all terms used have their ordinary meanings in the art, which will be understood by those skilled in the art. Reference is also made, for example, to standard manuals such as Sambrook et al. “Molecular Cloning: A Laboratory Manual” (2nd Edition), Volumes 1-3, Cold Spring Harbor Laboratory Press (1989); Lewin, “Genes IV,” Oxford University Press, New York (1990); and Roitt et al., “Immunology” (2nd Edition), Gower Medical Publishing, London, New York (1989), as well as the general prior art cited herein. Moreover, unless otherwise indicated, all methods, steps, techniques and operations not specifically described may be, and have been carried out in manners known per se, and the manners will be known by those skilled in the art. Reference is also made, for example, to standard manuals, the above general prior art and other references cited therein. As used herein, the term “and/or” covers all combinations of items linked by the term, and it should be considered that each combination has been individually listed herein. For example, “A and/or B” covers “A”, “A and B” and “B”. For example, “A, B and/or C” covers “A”, “B”, “C”, “A and B”, “A and C”, “B and C” and “A and B and C”.

When the term of “comprise” or “include” or “contain” is used herein to describe a sequence of a protein or nucleic acid, the protein or nucleic acid may consist of the sequence, or one or both ends of the protein or nucleic acid may have additional amino acids or nucleotides, but the activity described in the present invention still exists. Furthermore, it is clear to those skilled in the art that methionine encoded by an initiation codon at the N-terminal of a polypeptide will be retained under certain practical circumstances (for example, when expressed in a particular expression system), but the function of the polypeptide is not substantially affected. Therefore, when a particular polypeptide amino acid sequence is described in the specification and claims of the present invention, although the methionine encoded by the initiation codon at the N-terminal may not be contained, a sequence comprising the methionine is also covered in this regard, and accordingly, an encoding nucleotide sequence may also comprise the initiation codon; and vice versa.

The term “isolated” refers to a polypeptide or nucleic acid molecule that is considered “isolated” when it has been separated from at least one other component (e.g., another protein/polypeptide, another nucleic acid, another biological component or macromolecule, or at least one contaminant, impurity, or trace component) that is typically associated with it in its natural biological source and/or the reaction medium or culture medium from which the polypeptide or nucleic acid molecule it is obtained.

Specifically, a polypeptide or nucleic acid molecule is considered “isolated” when it has been purified at least 2-fold, particularly at least 10-fold, more particularly at least 100-fold, and up to 1000-fold or more. As determined by suitable techniques (e.g., appropriate chromatographic techniques such as polyacrylamide gel electrophoresis), the “isolated” polypeptide or nucleic acid molecule is preferably substantially homogeneous.

As used herein, “Synthetic T-cell Receptor and Antigen Receptor (STAR)” refers to a modified TCR in which the variable regions of the TCR are replaced with antibody variable regions or other receptor sequences, and the TCR constant regions may also be modified.

As used herein, the “antigen-binding region” (e.g., the antigen-binding region in STAR) means that it can specifically bind to the target antigen alone or in combination with another antigen-binding region.

The antigen-binding region may be derived from an antibody that specifically binds to the target antigen, including any commercially available antibody. The antigen-binding region may also be derived from a receptor that binds to a specific target protein.

As used herein, the term “antibody” refers to immunoglobulins and immunoglobulin fragments, whether naturally or partially or fully synthetically produced (e.g., recombinant), including any fragment that retains the binding specificity of full length immunoglobulin, including at least a portion of its variable region containing the immunoglobulin molecule. Therefore, antibodies include any protein having a binding domain homologous or substantially homologous to the antigen-binding region (antibody-binding site) of an immunoglobulin. Antibodies include antibody fragments. As used herein, the term antibody includes synthetic antibodies, recombinantly produced antibodies, multispecific antibodies (e.g., bispecific antibodies), human antibodies, non-human antibodies, camelid antibodies, single-domain antibodies, humanized antibodies, chimeric antibodies, intracellular antibodies, and antibody fragments, including but not limited to Fab fragments, Fab′ fragments, F(ab′)2 fragments, Fv fragments, disulfide-linked Fv (dsFv), Fd fragments, Fd′ fragments, single-chain Fv (scFv), single-chain Fab (scFab), diabodies, anti-idiotypic (anti-Id) antibodies, or antigen-binding fragments of any of the foregoing antibodies. The antibodies described herein include any immunoglobulin type (e.g., IgG, IgM, IgD, IgE, IgA, and IgY), any class (e.g., IgG1, IgG2, IgG3, IgG4, IgA1, and IgA2), or subclass (e.g., IgG2a and IgG2b).

As used herein, the variable domain or variable region is a specific Ig domain of the heavy or light chain of an antibody, that contain amino acid sequences that vary between different antibodies. Each light chain and each heavy chain has a variable region VL (also denoted as V_{L}) and VH (or also denoted as V_{H}), respectively. The variable domains provide antigen specificity and are therefore responsible for antigen recognition. Each variable region contains CDRs and framework regions (FRs), with CDRs being part of the antigen-binding site.

As used herein, “hypervariable region,” “HV,” “complementarity-determining region,” “CDR,” and “antibody CDR” are used interchangeably to refer to one of several segments within each variable region that together form the antigen-binding site of an antibody. Each variable region contains 3 CDRs, designated CDR1, CDR2, and CDR3. For example, for a conventional 4-chain antibody, the light chain variable region contains 3 CDRs, designated VL CDR1, VL CDR2, and VL CDR3 (or LCDR1, LCDR2, and LCDR3); the heavy chain variable region domain contains 3 CDRs, designated VH CDR1, VH CDR2, and VH CDR3 (or HCDR1, HCDR2, and HCDR3). For camelid antibodies or single-domain antibodies, since they have only one variable region, they contain only 3 CDRs, designated CDR1, CDR2, and CDR3.

In the context of the present invention, the terms “single-domain antibody,” “nanobody,” “heavy-chain single-domain antibody,” “VHH,” “VHH domain,” “VHH antibody fragment,” and “VHH antibody” are used interchangeably.

A “single-domain antibody” is the variable domain of an antigen-binding immunoglobulin known as a “heavy-chain antibody” (i.e., an “antibody lacking light chains”) (Hamers-Casterman C, Atarhouch T, Muyldermans S, Robinson G, Hamers C, Songa E B, Bendahman N, Hamers R.: “Naturally occurring antibodies devoid of light chains”; Nature 363,446-448 (1993)). The term “VHH” is used to distinguish the variable region of heavy-chain antibodies from the heavy-chain variable region (denoted as “VH” herein) present in conventional four-chain antibodies sand the light-chain variable region (denoted as “VL” herein) present in conventional four-chain antibodies. VHH specifically binds to epitopes without the need for other antigen-binding regions (unlike VH or VL in conventional 4-chain antibodies, where epitopes are recognized by VL and VH together). VHH is a small, stable, and efficient antigen recognition unit formed by a single-domain.

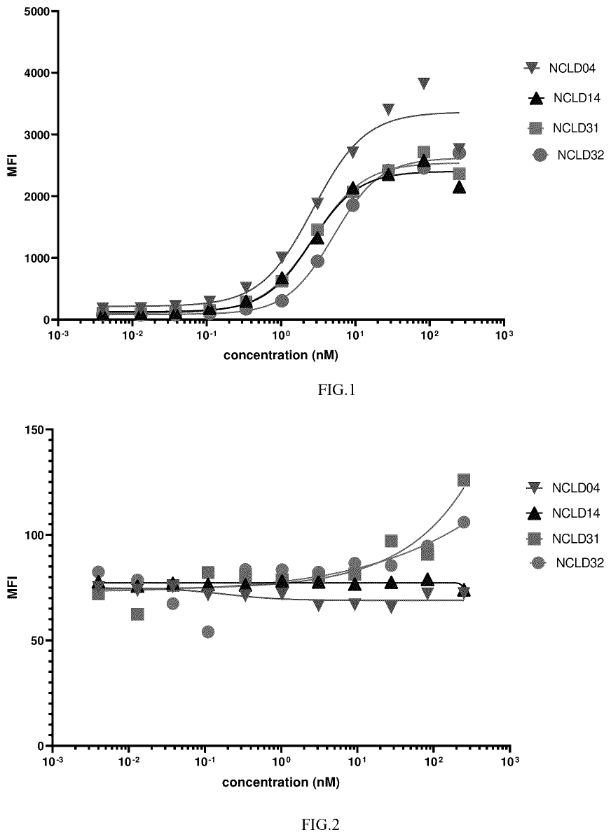

For example, as shown in FIG. 2 of Riechmann and Muyldermans, J. Immunol. Methods 231, 25-38 (1999), the amino acid residues of the VHH domain of camelids can be numbered according to the general numbering scheme for VH domains provided by Kabat et al. (Kabat et al., Sequences of Proteins of Immunological Interest, 5th Ed. Public Health Service, National Institutes of Health, Bethesda, Md. (1991)). However, alternative methods for numbering the amino acid residues of VH domains are known in the art and can also be similarly applied to VHH domains. For example, Chothia CDRs refer to the location of structural loops (Chothia and Lesk, J. Mol. Biol. 196:901-917 (1987)).

The AbM CDR represents a compromise between Kabat hypervariable regions and the Chothia structural loops and is used in Oxford Molecular's AbM antibody modeling software. The “Contact” CDRs is based on the analysis of available complex crystal structures. The CDRs of the single-domain antibodies of the present invention are determined according to the Kabat database.

VHH domains derived from camelids can be “humanized” (also referred to herein as “sequence optimization”, in addition to humanization, “sequence optimization” may also encompass other modifications to the sequence by providing one or more mutations that improve the properties of VHH, such as removing potential post-translational modification sites) by replacing one or more amino acid residues in the original VHH sequence with one or more amino acid residues present at corresponding positions in the VH domain of human conventional four-chain antibodies. Humanized VHH domains may contain one or more fully human framework region sequences. Humanization can be achieved using methods such as protein surface resurfacing and/or CDR grafting to a universal human framework.

In general, the term “specificity” refers to the number of different types of antigens or epitopes that a particular antigen-binding molecule or antigen-binding protein (e.g., the antibody of the present invention) can bind. The specificity of an antigen-binding protein can be determined based on its affinity and/or avidity. The affinity, expressed as the dissociation equilibrium constant (KD) of the antigen with the antigen-binding protein, is a measure of the binding strength between the epitope and the antigen-binding site on the antigen-binding protein: the smaller the KD value, the stronger the binding between the epitope and the antigen-binding protein (or, affinity can also be expressed as the association constant (KA), which is 1/KD). As will be understood by those skilled in the art, depending on the specific antigen of interest, affinity can be determined in a known manner. Avidity is a measure of the binding strength between an antigen-binding protein (e.g., an antibody) and its related antigen.

Avidity is related to both the affinity between the antigen and the antigen-binding site on the antigen-binding protein and the number of related binding sites present on the antigen-binding protein.

As used herein, “the amino acid number refers to SEQ ID NO: x” (SEQ ID NO: x is a specific sequence listed herein) means that the position number of the specific amino acid described is the position number of the corresponding amino acid on SEQ ID NO: x. The correspondence of amino acids in different sequences can be determined according to sequence alignment methods well known in the art. For example, the correspondence of the amino acids can be determined by an online alignment tool from EMBL-EBI (https://www.ebi.ac.uk/Tools/psa/), wherein two sequences can be aligned by using a Needleman-Wunsch algorithm by using default parameters. For example, an alanine at position 46 from its N-terminal of a polypeptide aligns with the amino acid at position 48 of SEQ ID NO: x in a sequence alignment, then the amino acid in the polypeptide may also be described herein as “an alanine at position 48 of the polypeptide, and the amino acid position refers to SEQ ID NO: x”.

The proteins/polypeptides mentioned in the present invention may contain a signal peptide (or leader sequence) at the N-terminal. Those skilled in the art will understand that in cells, the signal peptide sequence can direct the protein/polypeptide to a specific location in the cell, such as the cell membrane, and it may be cleaved and not included in the final product. Exemplary signal peptides include, but are not limited to, the IgE signal peptide (SEQ ID NO:37), GM-CSF signal peptide (SEQ ID NO:38), bovine prolactin pre-signal peptide, and natural signal peptides of the mentioned proteins/polypeptides such as IL-15Rα signal peptide, IL-15 signal peptide, etc. These signal peptide sequences are known in the art or can be easily identified by those skilled in the art based on the existing knowledge in the field.

The “expression vector” of the present invention may be a linear nucleic acid fragment, a cyclic plasmid, a viral vector, or an RNA capable of translation (e.g., mRNA). In some preferred embodiments, the expression vector is a viral vector, such as a lentiviral vector.

As used herein, the term “operably linked” means that a regulatory element (e.g., but not limited to, a promoter sequence, a transcription termination sequence, etc.) is linked to a nucleic acid sequence (e.g., a coding sequence or an open reading frame) such that the nucleotide sequence transcription is controlled and regulated by the transcriptional regulatory element. Techniques for operably linking a regulatory element region to a nucleic acid molecule are known in the art.

The term “regulatory sequence” and “regulatory element” are used interchangeably to refer to a nucleotide sequence that is located upstream (5′ non-coding sequence), intermediate or downstream (3′ non-coding sequence) of a coding sequence and affect the transcription, RNA processing or stability or translation of the relevant coding sequence. An expression regulatory element refers to a nucleotide sequence that can control the transcription, RNA processing or stability, or translation of a nucleotide sequence of interest. A regulatory sequence may include, but is not limited to, a promoter, a translation leader sequence, an intron, an enhancer, and a polyadenylation recognition sequence. Suitable promoters include, but are not limited to, the PGK promoter, hEF1a-HTLV promoter (as shown in SEQ ID NO:36), and MND promoter (SEQ ID NO:35).

As use herein, “subject” refers to an organism that suffers from or is prone to suffer from a disease (e.g., cancer) that can be treated by the antibody, the cell, method, or pharmaceutical composition of the present invention. A non-limiting example includes human, cattle, rat, mouse, dog, monkey, goat, sheep, cow, deer, and other non-mammals. In some preferred embodiments, the subject is human. As used herein, a “pharmaceutically acceptable carrier” includes any and all physiologically compatible solvents, dispersion medium, coatings, antibacterial and antifungal agents, isotonic agents and absorption retarders, etc. Preferably, the carrier is suitable for intravenous, intramuscular, subcutaneous, parenteral, spinal, or epidermal administration (e.g., by injection or infusion).

As used herein, a “therapeutically effective amount” or “therapeutically effective dose” or “effective amount” refers to the amount of a substance, compound, material or cell that is at least sufficient to produce a therapeutic effect after administration to a subject. Therefore, it is an amount necessary to prevent, cure, improve, block or partially block the symptoms of disease or disorder. For example, an “effective amount” of the cell or pharmaceutical composition of the present invention may preferably result in a decrease in the severity of disorder symptoms, an increase in the frequency and duration of the asymptomatic period of the disorder, or the prevention of injury or disability as a result of suffering from the disorder. For example, for the treatment of tumor, an “effective amount” of the antibody, the cell, the expression vector or pharmaceutical composition of the present invention may preferably inhibit tumor cell growth or tumor growth by at least about 10%, preferably at least about 20%, more preferably at least about 30%, more preferably at least about 40%, more preferably at least about 50%, more preferably at least about 60%, more preferably at least about 70%, and more preferably at least about 80%, as compared to an untreated subject. The ability to inhibit tumor growth can be evaluated in an animal model system that may predict efficacy in a human tumor. Alternatively, it is possible to perform evaluation by examining the ability to inhibit the growth of tumor cells which may be determined in vitro by tests known to those skilled in the art.

### Single-Domain Antibody Specifically Binding to Claudin18.2

In one aspect, the present invention provides a single-domain antibody that specifically binds to Claudin18.2, comprising CDR1, CDR2, and CDR3 selected from CDR1, CDR2 and CDR3 in either SEQ ID NO:4 or 8. The CDRs can be Kabat CDRs, AbM CDRs, Chothia CDRs, or Contact CDRs. In some embodiments, the CDRs are Kabat CDRs.

In some embodiments, for the convenience of purification and/or labeling, the single-domain antibody that specifically binds to Claudin18.2 may further comprise one or more additional tag sequences. For example, the additional tag can be a His-tag (such as a 6×His-tag) or an Fc-tag, which is beneficial for the separation and purification of the polypeptide or for prolonging its in-vivo half-life. Those skilled in the art will understand that these additional tags will not substantially affect the binding ability of the antibody.

The single-domain antibody specifically binding to Claudin18.2 provided by the present invention can have a KD value for binding to Claudin18.2 of less than about 1×10⁻⁷ M, preferably less than about 1×10⁻⁸ M, more preferably less than about 1×10⁻⁹ M, even more preferably less than about 1×10⁻¹⁰ M, and even more preferably less than about 1×10⁻¹¹ M.

### Expression Vectors and Methods for Preparing Single-Domain Antibodies

In another aspect, the present invention provides an isolated nucleic acid molecule encoding the single-domain antibody that specifically binds to Claudin18.2 of the present invention. In some embodiments, the nucleotide sequence of the nucleic acid molecule is codon-optimized for the host cell used for expression. In some embodiments, the nucleic acid molecule of the present invention is operably linked to an expression regulatory element such as a promoter.

The present invention also provides an expression vector for expressing the single-domain antibody of the present invention, which contains the nucleic acid molecule encoding the single-domain antibody that specifically binds to Claudin18.2 as described herein.

The present invention also provides a host cell for producing the single-domain antibody of the present invention, which is transformed with the aforementioned nucleic acid molecule or expression vector.

As used herein, a “host cell” is a cell used to receive, maintain, replicate, and amplify a vector. Host cells can also be used to express the polypeptide encoded by the nucleic acid or vector. When the host cell divides, the nucleic acid contained in the vector is replicated, thereby amplifying the nucleic acid.

Host cells can be eukaryotic cells or prokaryotic cells. Suitable host cells include, but are not limited to, CHO cells, various COS cells, HeLa cells, HEK cells such as HEK 293 cells.

In another aspect, the present invention provides a method for producing the single-domain antibody that specifically binds to Claudin18.2 as described herein, comprising:
(i) Culturing the host cell of the present invention under conditions suitable for the expression of the nucleic acid molecule or expression vector, and
(ii) Isolating and purifying the single-domain antibody that specifically binds to Claudin18.2 expressed by the host cell.

The methods and reagents for recombinantly producing polypeptides, such as specific suitable expression vectors, transformation or transfection methods, selection markers, methods for inducing protein expression, culture conditions, etc., as well known in the art. Similarly, the protein separation and purification techniques applicable to the method for producing the single-domain antibody that specifically binds to Claudin18.2 of the present invention are well-known to those skilled in the art.

However, the single-domain antibody that specifically binds to Claudin18.2 of the present invention can also be obtained by other methods known in the art for producing proteins, such as chemical synthesis, including solid-phase or liquid-phase synthesis.

STAR Targeting Claudin18.2 In some aspects, the present invention provides a synthetic T-cell receptor and antigen receptor (STAR) targeting Claudin18.2, which contains an antigen-binding region that specifically binds to Claudin18.2.

In some embodiments, the synthetic T-cell receptor and antigen receptor (STAR) targeting Claudin18.2 comprises a first peptide chain and a second peptide chain. wherein the first peptide chain contains a first constant region, the second peptide chain contains a second constant region, and the first peptide chain and/or the second peptide chain further contains an antigen-binding region that specifically binds to Claudin18.2. The first or second constant region is selected from the constant region of the TCRα chain or the TCRβ chain, respectively.

As used herein, “exogenous” means a protein or nucleic acid sequence from a foreign species, or if from the same species, a protein or nucleic acid sequence that has been significantly altered in composition and/or location from its natural form through deliberate human intervention.

The target-binding region refers to the region in an antigen receptor (such as STAR or CAR) that is used to specifically bind to a target antigen. The target-binding region can comprise one or more antigen-binding regions. The antigen-binding regions bind to a single protein region or epitope (antigenic determinant) of an antigen through antigen-antibody interactions or receptor-ligand interactions. In some embodiments, the antigen-binding region is an antibody or an antigen-binding fragment of an antibody, such as a single-domain antibody, scFv, or nanobody, etc.

As used herein, the “exogenous intracellular functional domain” can be the endodomain of a co-stimulatory molecule such as CD40, OX40, ICOS, CD28, 4-1BB, CD27, CD137; it can also be the endodomain of a coinhibitory molecule, such as TIM3, PD1, CTLA4, LAG3; it can also be the endodomain of a cytokine receptor such as an interleukin receptor (such as the IL-2β receptor, IL-7α receptor, or IL-21 receptor), an interferon receptor, a tumor necrosis factor superfamily receptor, a colony-stimulating factor receptor, a chemokine receptor, a growth factor receptor, or other membrane-protein; or it can be the domain of an intracellular protein such as NIK.

In some preferred embodiments, the exogenous intracellular functional domain is the endodomain of a co-stimulatory molecule, preferably the endodomain of OX40. In some embodiments, the endodomain of OX40 comprises the amino acid sequence shown in SEQ ID NO:23.

### Chimeric Antigen Receptor (CAR) Targeting Claudin18.2

In some aspects, the present invention provides a chimeric antigen receptor (CAR) targeting Claudin18.2, which contains an extracellular antigen-binding region (antigen-binding domain). The extracellular antigen-binding domain contains a Claudin18.2 single-domain antibody. The CAR, from the N-terminal to the C-terminal, sequentially comprises an extracellular antigen-binding region, a hinge region, a transmembrane domain, a co-stimulatory domain, and an intracellular signaling-transduction domain.

The hinge region is the region in a chimeric antigen receptor (CAR) that connects the extracellular antigen-binding region and the transmembrane domain. The hinge region is usually composed of a flexible peptide chain, which is used to provide spatial freedom and enhance the efficiency of antigen-binding.

The transmembrane domain is the region in a chimeric antigen receptor (CAR) that anchors the CAR in the cell membrane. The transmembrane domain is usually composed of hydrophobic amino acids which can stably embed in the cell membrane.

The intracellular signaling-transduction domain is the region in a chimeric antigen receptor (CAR) that is used to transmit activation signals. It usually contains immunoreceptor tyrosine-based activation motifs (ITAM) or other signaling motifs. These domains can activate the immune response of T cells.

In some embodiments, the CAR further comprises a transmembrane domain, such as the CD8α transmembrane domain or the CD28 transmembrane domain, preferably the CD8α transmembrane domain.

In some embodiments, the CAR further includes a hinge region located between the extracellular antigen-binding region and the transmembrane domain. For example, the hinge region is the CD8a hinge region.

In some embodiments, the CAR further contains a signal-transduction domain, such as a signal-transduction domain that can be used for T-cell activation, for example, the signal-transduction domains selected from signal-transduction domains of TCRζ, FcRγ, FcRβ, FcRε, CD3γ, CD3δ, CD3ε, CD3ζ, CD5, CD22, CD79a, CD79b, and CD66d. In some preferred embodiments, the CAR contains the CD3ζ signal-transduction domain.

In some embodiments, the CAR further contains one or more co-stimulatory domains, such as the co-stimulatory domains selected from co-stimulatory domains of CD3, CD27, CD28, CD83, CD86, CD127, 4-1BB, and 4-1BBL.

In some embodiments, the CAR, in the direction from the N-terminus to the C-terminus, contains the extracellular antigen-binding region, the hinge region, the transmembrane domain, the co-stimulatory domain, and the signal-transduction domain. In some embodiments, the hinge region is the CD8α hinge region, the transmembrane domain is the CD8α transmembrane domain, the signal-transduction domain is the CD3ζ signal-transduction domain, and the co-stimulatory domain is the 4-1BB co-stimulatory domain.

In some embodiments, the extracellular antigen-binding region further contains an antigen-binding region that specifically binds to another antigen. Thus, the CAR can also target the other antigen. Preferably, the antigen-binding region that specifically binds to the other antigen comprises a single-chain antibody (scFv) or a single-domain antibody that specifically binds to the other antigen.

In some embodiments, the other antigen is CCR8. The antigen-binding region specifically binding to CCR8 comprises CDR1 as shown in SEQ ID NO:41, CDR2 as shown in SEQ ID NO:42, and CDR3 as shown in SEQ ID NO:43. In some embodiments, the other antigen is CCR8. The antigen-binding region specifically binding to CCR8 comprises the amino acid sequence shown in SEQ ID NO:44 (single-domain antibody).

In some embodiments, the other antigen is MSLN. The antigen-binding region specifically binding to MSLN comprises CDR1 as shown in SEQ ID NO:31, CDR2 as shown in SEQ ID NO:32, and CDR3 as shown in SEQ ID NO:33. In some embodiments, the other antigen is MSLN. The antigen-binding region specifically binding to MSLN comprises the amino acid sequence shown in SEQ ID NO:34 (single-domain antibody).

In some embodiments, the other antigen is LILRB4. Preferably, the antigen-binding region specifically binding to LILRB4 comprises CDR1 as shown in SEQ ID NO:45, CDR2 as shown in SEQ ID NO:46, and CDR3 as shown in SEQ ID NO:47. Preferably, the antigen-binding region specifically binding to LILRB4 comprises the amino acid sequence shown in SEQ ID NO:48 (single-domain antibody).

In some specific embodiments, the CAR comprises an amino acid sequence shown in any one of SEQ ID NO:69 to 73.

### Therapeutic Immune Cells

In another aspect, the present invention provides an isolated therapeutic immune cell, which comprises the STAR of the present invention.

In some embodiments, the immune cell is a T cell. In other embodiments, the immune cell is a natural killer (NK) cell.

In some embodiments, the STAR of the present invention is co-expressed with a membrane-bound IL-15 protein (mbIL-15) in the therapeutic immune cell.

mbIL-15 refers to a fusion protein formed by the connection (such as through a linker) of IL-15 and the extracellular domain of IL-15Rα. The exemplary amino acid sequence of IL-15 is shown in SEQ ID NO:27, but also includes amino acid sequences having at least 80%, at least 85%, at least 90%, at least 95%, and even at least 99% sequence identity with SEQ ID NO:27. The exemplary amino acid sequence of the extracellular domain of IL-15Rα is shown in SEQ ID NO:28, but also includes amino acid sequences having at least 80%, at least 85%, at least 90%, at least 95%, and even at least 99% sequence identity with SEQ ID NO:28. The exemplary amino acid sequence of the linker connecting the extracellular domain of IL-15Rα and IL-15 is shown in SEQ ID NO:29. The exemplary amino acid sequence of mbIL-15 is shown in SEQ ID NO:30, but also includes amino acid sequences having at least 80%, at least 85%, at least 90%, at least 95%, and even at least 99% sequence identity with SEQ ID NO:30.

The immune cells of the present invention, such as T cells, can be obtained from many non-limiting sources through various non-limiting methods, including peripheral blood mononuclear cells, bone marrow, lymph node tissue, umbilical cord blood, thymus tissue, ascites, pleural effusion, spleen tissue, and tumors. In some embodiments, the cells can be derived from healthy donors or patients diagnosed with cancer. In some embodiments, the cells can be part of a mixed population of cells with different phenotypic characteristics. For example, immune cells such as T cells can be obtained by isolating peripheral blood mononuclear cells (PBMC), followed by activation and expansion with specific antibodies.

In some embodiments, the immune cells such as T cells of the present invention are isolated (ex vivo) immune cells.

In some embodiments of various aspects of the present invention, the immune cells such as T cells are derived from autologous cells of a subject. As used herein, “autologous” means that the cells, cell lines, or cell populations used for treating a subject are derived from the subject. In some embodiments, the immune cells such as T cells are derived from allogeneic cells, for example, from a donor who is human leukocyte antigen (HLA)-compatible with the subject. Cells from a donor can be transformed into non-alloreactive cells using standard protocols and replicated as needed to obtain cells that can be administered to one or more patients.

In some embodiments, the therapeutic immune cells such as T cells are therapeutic immune cells such as T cells that can be obtained or have been obtained by the expression vectors or methods of the present invention described below.

### Expression Vectors and Methods for Preparing Therapeutic Immune Cells

In one aspect, the present invention provides an expression vector comprising the coding sequence of the STAR of the present invention.

In some embodiments, the expression vector further comprises the coding sequence of the membrane-bound IL-15 protein (mbIL-15) of the present invention.

The coding sequence in the expression vector of the present invention can be operably linked to regulatory elements such as a promoter for expression in cells.

In some embodiments, the mbIL-15 can be driven by a separate promoter for expression.

In some embodiments, the expression vector comprises:
a) A coding nucleotide sequence of a fusion polypeptide in which the first peptide chain of the STAR of the present invention and the second peptide chain of the STAR of the present invention are linked by a self-cleaving peptide;
b) A coding nucleotide sequence of a fusion polypeptide in which the first peptide chain of the STAR of the present invention, the second peptide chain of the STAR of the present invention, and the mbIL-15 of the present invention are linked by a self-leaving peptide; or
c) A coding nucleotide sequence of a fusion polypeptide in which the mbIL-15 of the present invention is linked by a self-cleaving peptide.

As used herein, the “self-cleavage peptide” means a peptide that can achieve self-cleavage in a cell. For example, the self-cleavage peptide may contain a protease recognition site so as to be recognized and specifically cleaved by proteases in a cell. Alternatively, the self-cleavage peptide may be a 2A polypeptide. The 2A polypeptide is a kind of short peptide from virus, and its self-cleavage occurs during translation. When two different target proteins are linked by 2A polypeptide and expressed in the same reading frame, the two target proteins are generated almost in a ratio of 1:1. A common 2A polypeptide may be P2A from porcine techovirus-1, T2A from Thosea asigna virus, E2A from equine rhinitis A virus, and F2A from foot-and-mouth disease virus. Among them, P2A has the highest cleavage efficiency and is therefore preferred. A variety of functional variants of these 2A polypeptides are also known in the art, which can also be used in the present invention. 2A polypeptides can also be combined with a Furin recognition sequence to remove additional introduced amino acid sequences.

In some embodiments, the self-cleaving peptide is a 2A polypeptide, such as the P2A polypeptide. In some embodiments, the self-cleaving peptide is a Furin-2A polypeptide, such as the Furin-P2A polypeptide shown in SEQ ID NO:26.

In some embodiments, the different parts of the fusion polypeptide can be arranged in different ways as long as they are separated by a self-cleaving peptide. For example, in some embodiments, the fusion polypeptide can comprise, in the direction from the N-terminal to the C-terminal, the second peptide chain (the peptide chain containing the constant region of the TCRβ chain), a self-cleaving peptide such as Furin-P2A, and the first peptide chain (the peptide chain containing the constant region of the TCRα chain). In some embodiments, the fusion polypeptide can comprise, in the direction from the N-terminal to the C-terminal, the second peptide chain, a self-cleaving peptide such as Furin-P2A, the first peptide chain, a self-cleaving peptide such as Furin-P2A, and the mbIL-15. When there are multiple self-cleaving peptides, the self-cleaving peptides can be the same or different.

In another aspect, the present invention provides a method for preparing therapeutic immune cells, comprising:
Step 1) Providing starting immune cells;
Step 2) Introducing the expression vector of the present invention into the starting immune cells; and
Step 3) Harvesting the immune cells obtained in Step 2).

In some embodiments, the starting immune cells are T cells. In other embodiments, the starting immune cells are NK cells.

The starting immune cells of the present invention, such as T cells, can be obtained from many non-limiting sources through various non-limiting methods, including peripheral blood mononuclear cells, bone marrow, lymph node tissue, umbilical cord blood, thymus tissue, ascites, pleural effusion, spleen tissue, and tumors. In some embodiments, the cells can be derived from healthy donors or patients diagnosed with cancer. In some embodiments, the cells can be part of a mixed population of cells with different phenotypic characteristics. For example, starting immune cells such as T cells can be obtained by isolating peripheral blood mononuclear cells (PBMCs) and then activating and expanding them with specific antibodies.

In some embodiments, the starting immune cells, such as T cells, are isolated (ex-vivo) immune cells, such as T cells. Therefore, the therapeutic immune cells, such as T cells, obtained by the present invention are also isolated (ex-vivo) therapeutic immune cells, such as T cells.

In some embodiments, the method of the present invention is an in-vitro method.

In some embodiments of various aspects of the present invention, the starting immune cells, such as T cells, are derived from the autologous cells of a subject. As used herein, “autologous” means that the cells, cell lines, or cell populations used for treating a subject are derived from the subject. In some embodiments, the starting immune cells, such as T cells, are derived from allogeneic cells, for example, from a donor who is human leukocyte antigen (HLA)-compatible with the subject. Standard protocols can be used to convert the cells from the donor into non-alloreactive cells and be replicated them as needed to produce cells that can be administered to one or more patients.

The introduction of the expression vector into immune cells, such as T cells, can be performed using by methods known in the art, including but not limited to microinjection, electroporation, virus-mediated transfection, liposome-mediated transfection, etc.

In some embodiments, the method further includes, between Step 2) and Step 3), Step x: Expand the immune cells, such as T cells, obtained in Step 2). Immune cells, such as T cells, may be expanded using methods known in the art.

In some embodiments, the method further comprises Step y: Screen for immune cells, such as T cells, that express the STAR or CAR, or immune cells, such as T cells, that co-express the mbIL-15 and the STAR or CAR. In some embodiments, Step y) can be performed after Step 2). In some embodiments, Step y) can be carried out after Step 2) and before Step x). In some embodiments, Step y) can be performed after Step x). In some embodiments, the screening is performed by flow cytometry.

In another aspect, the present invention provides therapeutic immune cells, such as T cells, that can be obtained or are obtained by the expression vector or the method of the present invention.

### Pharmaceutical Compositions and Applications

In another aspect, the present invention provides a pharmaceutical composition, which comprises the single-domain antibody of the present invention, the STAR of the present invention, the CAR of the present invention, the therapeutic immune cells of the present invention, and/or the expression vector of the present invention, and a pharmaceutically acceptable carrier.

In another aspect, the present invention provides the use of the single-domain antibody of the present invention, the STAR of the present invention, the CAR of the present invention, the therapeutic immune cells of the present invention, the expression vector of the present invention, and/or the pharmaceutical composition of the present invention in the preparation of a medicament for treating a disease in a subject.

In another aspect, the present invention provides a method for treating a disease in a subject. The method includes administering to the subject a therapeutically effective amount of the single-domain antibody of the present invention, the therapeutic immune cells of the present invention, the expression vector of the present invention, and/or the pharmaceutical composition of the present invention. In practical applications, the dosage levels of the antibody, cells, or expression vector in the pharmaceutical composition of the present invention may vary to obtain the amount of active ingredient that can achieve the desired therapeutic response for a specific patient, the composition, and the mode of administration, without toxicity to patients. The selected dosage level depends on various pharmacokinetic factors, including the activity of the specific composition of the present invention, the route of administration, the time of administration, the excretion rate of the specific compound used, the duration of treatment, other drugs, compounds, and/or materials used in combination with the specific composition, the age, gender, weight, condition, general health, and medical history of the patient being treated, as well as other similar factors well-known in the medical field.

The antibody, expression vector, therapeutic immune cells, pharmaceutical composition, or medicament according to the present invention can be administered in any convenient way, including injection, infusion, implantation, or transplantation. The antibody, expression vector, therapeutic immune cells, or pharmaceutical composition described herein can be administered via intravenous, intralymphatic, intradermal, intratumoral, intramedullary, intramuscular, or intraperitoneal routes. In one embodiment, the antibody, expression vector, therapeutic immune cells, or pharmaceutical composition of the present invention is preferably administered by intravenous injection.

In the embodiments of various aspects of the present invention, the disease is a Claudin18.2-related tumor, preferably hematologic tumor, or solid tumor; Preferably, the tumor is selected from the following group: lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, digestive tract cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma; More preferably, the tumor is selected from the following group: rectal cancer, breast cancer, gastric cancer, metastatic brain cancer, metastatic liver cancer, lung cancer, digestive tract cancer, mesothelioma, pancreatic cancer, ovarian cancer, or B-cell malignancies.

### EXAMPLES

The present invention will be further described below in conjunction with specific embodiments. However, these specific embodiments should not be construed as limiting the protection scope of the present invention. Those skilled in the art can make various changes or modifications to these specific embodiments without departing from the scope of the technical solution of the present invention, and the modified and changed embodiments still fall within the protection scope of the present invention.

The lentiviral vectors and lentiviral packaging plasmids used in the examples of the present application were purchased from commercial companies or synthesized by commercial companies. The gene fragments used in the examples of the present application, including signal peptides, antibody binding regions, hinge regions, TCR constant regions, tag proteins, etc., were synthesized from commercial companies. The embodiments of the present application are only for further describing the present invention and are not intended to limit the scope of the present invention.

### Optimization of STAR

The secreted antibody (Antibody, Ab) or B-cell receptor (BCR) produced by B cells has great similarity to the T-cell receptor (TCR) in terms of genetic structure, protein structure and spatial conformation. Both the antibody and TCR consist of a variable region and a constant region, in which the variable region plays the role of antigen-recognizing and binding, while the constant region domain plays the role of structural interaction and signal transduction. By replacing the variable regions of TCR α and β chains (or TCR γ and δ chains) with the heavy chain variable region (VH) and light chain variable region (VL) of the antibody, VHH or scFv, an artificially synthetic chimeric molecule called Synthetic T-Cell Receptor and Antibody Receptor (STAR) can be constructed.

In some embodiments, a STAR molecule has two chains, wherein the first chain is obtained by fusing an antigen recognition sequence (such as an antibody heavy chain variable region, VH) with a constant region (C α) of a T cell receptor α chain (TCR α), and the second chain is obtained by fusing an antigen recognition sequence (such as an antibody light chain variable region, VL) with a constant region (C β) of a T cell receptor β chain (TCR β). The antigen recognition domain (such as VH, VL or scFv, etc.) and the constant domain (constant domain of TCR α, β, γ and δ) in the construct can be arranged and combined to form a variety of constructs with different configurations but similar functions. The first and second chains of STAR molecule, after expressing in T cells, will combine with endogenous CD3 εδ, CD3γε c and CD3 ζζ chains in the endoplasmic reticulum to form a eight-subunit complex, which is present on the surface of cell membrane in the form of complex. An immunoreceptor tyrosine-based activation motif (ITAM) is a signal transduction motif in a TCR molecule, with its conserved sequence of YxxL/V. The endodomains of CD3 ε, δ, γ and ε chains comprise one ITAM sequence, and that of CD3 ζ chain comprises three ITAM sequences, so a complete STAR complex has a total of ten ITAM sequences. When the antigen recognition sequence of a STAR receptor binds to its specific antigen, the intracellular ITAM sequence will be phosphorylated successively, which then in turn activate the downstream signaling pathway, activating transcription factors such as NF-κβ, NFAT, and AP-1, etc., to initiate the activation of T cells and produce effector functions.

Due to the constant region sequences of human, primate and murine TCR α/β chains (mouse TCRAC/mouse TCRBC) are highly conserved, and have the same key amino acid sequence as well, they can be replaced with each other, increasing the efficiency of correct pairing of STAR molecules, reducing the possibility of mispairing leading to unknown specificities, and increasing the safety. The inventors of the present invention have previously modified the constant region of STAR, such as cysteine substitution and transmembrane-domain hydrophobic amino acid substitution, to improve its performance.

1) Modifications of Constant Regions Derived from Mice

For the constant-region sequences of mouse-derived TCRα/β chains, humanization modification, cysteine substitution, and transmembrane-domain hydrophobic amino acid substitution can be carried out.

Introducing Cysteine Point Mutations to Form Disulfide Bonds: the amino acid threonine (T) at position 48 is mutated to Cysteine (C) which is derived from a wild-type mouse TCRα chain constant region, and the amino acid serine (S) at position 56 is mutated to cysteine (C) which is derived from a wild-type mouse TCRβ chain constant region. The two newly added cysteines will form a disulfide bond between the two chains of STAR, thereby reducing the mispairing of the two chains of STAR with endogenous TCR chains and helping the STAR molecule form a more stable complex. The obtained α-chain constant region is named TRAC(Cys), and the obtained β-chain constant region is named TRBC(Cys).

Design of Hydrophobic Amino Acid Substitution in the STAR Transmembrane Region:
three amino acid mutations were carried out at amino acid positions 111 to 119 in the transmembrane domain of TCR α chain constant region: serine (S) at position 112 was mutated to leucine (L), methionine (M) at position 114 was mutated to isoleucine (I), and glycine (G) at position 115 was mutated to serine (V). The whole amino acid sequence in this region was changed from LSVMGLRIL (SEQ ID NO:74) to LLVIVLRIL(SEQ ID NO:75). This design increased the hydrophobicity of transmembrane domain, counteracts the instability caused by positive charges carried by the TCR transmembrane domain, and makes STAR molecule more stable on the cell membrane, thus obtaining better functions. The α-chain constant region obtained by combining the cysteine mutation and the hydrophobic-region mutation is named TRAC(Cys-TM), and the corresponding R-chain constant region is named TRBC(Cys-TM), wherein TRBC(Cys-TM) is the same as TRBC(Cys).

To further optimize the design of the STAR molecule, a specific rearrangement is carried out on the N-terminal of the STAR molecule's constant region based on the cysteine point mutation of the mouse-derived constant region and the hydrophobic amino acid mutation of the α-chain constant region to obtain better results. Rearrangement means partial sequence deletion and simultaneous humanization mutation of some sequences. The significance of the humanized mutation is to minimize non-human sequences in the STAR molecule while ensuring the function of the STAR molecule to circumvent the possibility that STAR-T cells are rejected by receptors in clinical applications to the greatest extent. Therefore, the 18 amino acids at the N-terminal of the TCR α chain constant region are modified, including the 6th amino acid E is replaced by D, the 13th amino acid K is replaced by R, and the amino acids at positions 15-18 are deleted. The obtained α-chain constant region is named TRAC(Nrec-Cys-TM). Further, the 25 amino acids at the N-terminal of the TCR β chain constant region are modified, including the 3rd amino acid R is replaced by K, the 6th amino acid T is replaced by F, the 9th amino acid K is replaced by E, the 11th amino acid S is replaced by A, the 12th amino acid L is replaced by V, and the amino acids at positions 17, 21-25 are deleted. The obtained R-chain constant region is named TRBC(Nrec-Cys-TM).

The modified TCRα chain constant region is derived from the TCRα chain constant region of rodents (preferably mice, more preferably mouse), and compared with the wild-type TCRα chain constant region of rodents (preferably mice, more preferably mouse), wherein the substitution of the 122nd amino acid K replaced by R. The modified TCRβ chain constant region is derived from the TCRβ chain constant region of rodents (preferably mice, more preferably mouse), wherein the lysine at positions 150, 168, or 170 is replaced by arginine.

In addition, co-stimulatory molecule such as the cytoplasmic region of OX40 can be connected to the C-terminal of the α chain constant region and/or the 3 chain constant region to further enhance the function of STAR. The co-stimulatory molecule can be connected to the C-terminal of the α chain constant region and/or the 3 chain constant region through a linker, such as a (G4S)₃ linker. The constant region connected to the co-stimulatory molecule, in addition to the above-mentioned modifications, can also lack the natural intracellular region compared to the wild-type constant region, which further improves the function of STAR. For example, the α chain constant region can lack the amino acids at positions 136-137; and/or the 3 chain constant region can lack the amino acids at positions 167-172.

2) Modifications of Constant Regions Derived from Humans

For the constant-region sequences of human-derived TCRα/β chains, one or more of minimal murinization modification, cysteine substitution, transmembrane-domain hydrophobic amino acid substitution, or rearrangement can be carried out, as follows: Cysteine Point Mutation in the Human-Derived Constant Region of STAR (hereinafter referred to as hcSTAR): the amino acid threonine (T) at position 47 is mutated to cysteine (C) which is derived from a wild-type human TCRα chain constant region, and the amino acid serine (S) at position 56 is mutated to cysteine (C) which is derived from a wild-type human TCRβ chain constant region (this mutation is named Cys2). An additional disulfide bond is formed between the α chain constant region and the 3 chain constant region of the STAR molecule, reducing the mispairing of the two chains of STAR with endogenous TCR chains, helping the STAR molecule form a more stable complex, and thus obtaining better functions.

Minimal Murinization (abbreviated as MM) of the Human-Derived Constant Region of STAR: In the constant region of the TCRα chain, the 90th P is mutated to S, the 91st E is mutated to D, the 92nd S is mutated to V, and the 93rd S is mutated to P. In the constant region of the TCRβ chain, the 17th E is mutated to K, the 21st serine (S) is mutated to A, the 132nd F is mutated to I, the 135th E is mutated to A, and the 138th Q is mutated to H.

Hydrophobic Amino Acid Substitution (abbreviated as TM) in the STAR Transmembrane Region: Two amino acid site mutations are carried out at the amino acid positions 110 to 118 in the transmembrane domain of the TCRα chain constant region. The 115th serine (S) is mutated to leucine (L), and the 118th glycine (G) is mutated to valine (V). This design increases the hydrophobicity of the transmembrane region, counteracts the instability caused by the positive charge carried by the TCR transmembrane region, enables the STAR molecule to exist more stably on the cell membrane, and thus obtains better functions.

In addition, co-stimulatory molecule such as the cytoplasmic region of OX40 can be connected to the C-terminal of the α chain constant region and/or the β chain constant region to further enhance the function of STAR. The co-stimulatory molecule can be connected to the C-terminal of the α chain constant region and/or the β chain constant region through a linker, such as a (G4S)₃ linker. The constant region connected to the co-stimulatory molecule, in addition to the above-mentioned modifications, can also lack the natural intracellular region compared to the wild-type constant region, which further improves the function of STAR.

In the existing technologies, in order to kill tumors while improving the tumor immunosuppressive microenvironment, patients need to receive separate treatments or take different medications. This not only affects the therapeutic effect of tumor treatment, increases the burden on patients, but also raises the risk of adverse drug reactions. The dual-functional STAR-T cells of the present invention fully utilize their natural multi-chain structure. By attaching an antibody portion targeting a receptor (such as CCR8 or LILRB4) on immunosuppressive immune cells to one chain and an antibody portion targeting tumor antigens (such as MSLN or Claudin18.2) to the other chain, they achieve dual functions. Due to the structural advantage, the two antibody fragments do not interfere with each other and can function independently. Moreover, the antibody fragment targeting the receptor on immunosuppressive immune cells can eliminate the immunosuppressive effect of Treg cells and improve the tumor immunosuppressive microenvironment, thereby enhancing the tumor-killing ability of the antibody fragment targeting tumor antigens. In animal models of epithelial cell carcinoma, glioblastoma, and liver cancer, the dual-functional STAR-T cells of the present invention exhibit better anti-tumor effects than traditional CAR-T cells and have no obvious toxic and side effects. The dual-functional STAR-T cells of the present invention can achieve the effect of killing tumors and improving the tumor immunosuppressive microenvironment simultaneously in the form of a single drug.

### Example 1: Screening of Nanobodies Targeting Claudin18.2

1.1 Immunization of Alpacas with Human Claudin18.2 Protein

Healthy alpacas were immunized with the commercially available extracellular region of human Claudin18.2 protein (100 μg) (purchased from ACRO Biosystems, product number CL2-H52P7). The adjuvants included complete Freund's adjuvant (CFA, Sigma) and incomplete Freund's adjuvant (IFA, Sigma). The extracellular region of the human Claudin18.2 protein, which was expressed and purified as described above, was diluted with PBS and then mixed with the corresponding adjuvant at a ratio of 1:1. The antigen and the adjuvant were thoroughly mixed to form a stable emulsion. The antigen mixture was drawn into a syringe and injected subcutaneously at multiple points on the neck skin of the alpacas four times (immunization injections were carried out on days 1, 14, 28, and 42 respectively), with 100-200 μL injected at each point and 100 μg of Claudin18.2 antigen each time.

On day 53, alpaca blood samples were collected from the ear marginal vein, and the serum was extracted for antibody titer detection. The P/N value of the serum diluted 200,000-fold was greater than 2.

On days 54, 57, and 60: 30-40 mL of alpaca blood samples were collected from the hind-leg veins of the alpacas for PBMC isolation.

### 1.2 PBMC Isolation, RNA Extraction, Reverse Transcription, and Phage Library Construction

PBMCs were isolated from the alpaca blood samples obtained in the previous step using Ficoll separation solution (Cytiva, product number 17544202) according to conventional procedures. RNA was isolated from the PBMCs using conventional methods, and cDNA was synthesized using the Invitrogen reverse-transcription kit (Thermo Scientific, product number #K1622).

The VHH sequences were obtained through two rounds PCR, and homologous arms of the phagemid vector were added to both ends of the sequence. The VHH fragments were ligated to the phagemid vector, and then the ligation product was concentrated and purified (gel extraction kit, TIANGEN, product number #matched with 19-03). The purified ligation product was electro-transformed into competent Escherichia coli cells. Helper phages were added for infection and further cultivation. The phages were harvested and concentrated, and their titers were determined.

1.3 Antibody Screening from the Phage Library

The phage library obtained in the above steps was subjected to three rounds of antibody screening, with each round including one positive selection and one negative selection. First, the phages were incubated with antigen peptides, and the phages that could not bind were discarded, and the phages that bound to the antigen peptide were retained. Then, the retained phages were incubated with BSA for negative selection, and the phages that did not bind to BSA were retained.

The phages obtained after three rounds of screening and M13KO7 helper phages were used to co-infect TG1 cells, The infected cells were plated on 2YT-AK plates. Single colonies were picked for phage expansion. The phages were harvested for binding detection to determine the available phages/antibodies. The four obtained antibodies were named NCLD04, NCLD14, NCLD31, NCLD32 respectively.

### Example 2: Performance Detection of Nanobodies Targeting Claudin18.2

### 2.1. BLI Antibody Affinity of Claudin18.2 Nanobodies

The affinity of Claudin18.2 nanobodies was determined. The affinity of the antibodies was measured using the Fortebio Octet® detection instrument based on the biolayer interferometry (BLI) technique.

After the 5 antibodies were immobilized on the Protein A biosensor, the antibodies could bind to the Human Claudin18.2 His tag protein. The measured affinity was calculated using Data Analysis 11 software of the Octet RED96e (ForteBio). The results are shown in Table 1. The affinities (KD values) of antibody NCLD04 and antibody NCLD31 for the Claudin18.2 protein are both favorable. The antibody sequences are shown in Table 2.

TABLE 1
Detection of BLI Affinity of Claudin18.2 Nanobodies

| VHH-IgG1 Fc | KD (M) | Kon (1/Ms) | Kdis (1/s) |
|---|---|---|---|
| NCLD04 | 2.74E−10 | 7.35E+05 | 2.01E−04 |
| NCLD14 | 7.26E−08 | 6.29E+04 | 4.56E−03 |
| NCLD31 | 6.80E−10 | 8.84E+05 | 5.87E−04 |
| NCLD32 | 1.75E−09 | 5.04E+05 | 8.80E−04 |

TABLE 2
Sequences of Claudin18.2 Nanobodies

| | | | SEQ |
|---|---|---|---|
| Antibody | Description | Sequence | ID NO |
| NCLD04 | CDR1 | GLTFNDYA | 1 |
| | CDR2 | IRGSGGVT | 2 |
| | CDR3 | NARPLFGWSEEY | 3 |
| | VHH | DVQLQESGGGLVQPGGSL | 4 |
| | | RLSCTASGLTFNDYAVSW | |
| | | LRQAPGKEREFVATIRGS | |
| | | GGVTNYGASAEGRFIISR | |
| | | ENAENTVYLQMNSLKSED | |
| | | TAVYYCNARPLFGWSEEY | |
| | | WGQGTQVTVSS | |
| NCLD14 | CDR1 | GFTFSTYN | 5 |
| | CDR2 | IKWSASTT | 6 |
| | CDR3 | AGSPHIALGTRTEMYPY | 7 |
| | VHH | DVQLQESGGGLVQPGGSL | 8 |
| | | RLSCATSGFTFSTYNMGW | |
| | | FRQAPGKEREFVGSIKWS | |
| | | ASTTYYADSVKGRFTISR | |
| | | DNAKNTVYLQMNSLKPED | |
| | | TAIYYCAGSPHIALGTRT | |
| | | EMYPYWGQGTQVTVSS | |

### 2.2 EC50 Affinity of Claudin18.2 Nanobody

The EC50 value of the affinity of the Claudin18.2 nanobody was determined. SNU1-hClaudin18.2 cells were cultured until the cell density reached 80%, and then the experiment was started. The cells were added to 96-well plates at a density of 1×10⁶ cells/well with a volume of 100 μL for flow cytometry staining. The antibodies were serially diluted at a starting concentration of 750 nM with a 3-fold dilution ratio, and 11 samples were prepared for each antibody. The prepared antibodies were added to the cells in the 96-well plates, and after staining at 4° C. for 30 min, the antibodies were washed off with PBS. Then, a secondary antibody was added for staining: APC anti-human IgG Fe Antibody (Cat: 410711, Lot: B343074), and the secondary antibody was diluted 1:200. After the staining was completed, the APC fluorescence value was detected by flow cytometry, and draw a curve based on the experimental results and calculate the EC50 value.

As shown in FIG. 1 and Table 3, the antibody corresponding to lower EC50 values indicate higher antibody affinity. Antibody NCLD14 has a high EC50 value, indicating that the antibody has a relatively poor affinity. In contrast, the other antibodies, NCLD04, NCLD31, and NCLD32, exhibit low EC50 values, signifying that these antibodies have higher affinities. Among these, antibody NCLD04 demonstrates the best affinity.

TABLE 3
EC50 Affinity of Antibodies against Claudin18.2

| Antibody | EC50(nM) |
|---|---|
| NCLD04 | 2.686 |
| NCLD14 | 20.99 |
| NCLD31 | 2.744 |
| NCLD32 | 5.073 |

### 2.3. EC50 Antibody Specificity of Claudin18.1 Nanobody

The EC50 value of the affinity of the Claudin18.1 nanobody was determined. SNU1-hClaudin18.1 cells were cultured until the cell density reached 80%, and then the experiment was started. The cells were added to 96-well plates at a density of 1×10⁶ cells/well with a volume of 100 μL for flow cytometry staining. The antibodies were serially diluted at a starting concentration of 750 nM with a 3-fold dilution ratio, and 11 samples were prepared for each antibody. The prepared antibodies were added to the cells in the 96-well plates, and after staining at 4° C. for 30 min, the antibodies were washed off with PBS. Then, a secondary antibody was added for staining: APC anti-human IgG Fc Antibody (Cat: 410711, Lot: B343074), and the secondary antibody was diluted 1:200. After the staining was completed, the APC fluorescence value was detected by flow cytometry, and draw a curve based on the experimental results and calculate the EC50 value.

As shown in FIG. 2, the above-detected antibody NCLD04 did not bind to SNU1-hClaudin18.1 cells. Among them, the MFI (Mean Fluorescence Intensity) of antibody NCLD14 increased slightly, and the MFI of the antibodies NCLD31 and NCLD32 increased more significantly.

### 2.4. BLI Epitope Analysis of Claudin18.2 Nanobodies

The competitive binding of antibodies was detected by the Bio-Layer Interferometry (BLI) with the Fortebio Octet® detection instrument. The stationary phase was the Claudin18.2 protein, and the mobile phase was the NCLD nanobodies (antibodies formed by fusing the NCLD nanobodies with the Fe region, including NCLD04, NCLD14, NCLD31 and NCLD32). The results showed that competitive binding existed for all the antibodies.

### 2.5. MPA Specificity Detection of Claudin18.2 Nanobodies

The MPA developed and used by Integral Molecular is an array composed of more than 5,220 human membrane proteins (covering 94% of human membrane proteins). Each human membrane protein in the MPA has a complete structure and can be expressed in native conformation within living cells. MPA serves as in vitro tool that can rapidly and comprehensively screen the specificity of candidate therapeutic drugs.

As shown in FIG. 3, the antibody NCLD31 binds non-specifically to Claudin18.1, the antibody NCLD32 binds non-specifically to Claudin22, and the antibody NCLD04 specifically targets Claudin18.2 with no off-target binding.

### Example 3. Screening of the Efficacy of Claudin18.2 Antibody Sequences

### 3.1. Construction of Claudin18.2 STAR Expression Vector and Lentiviral Packaging

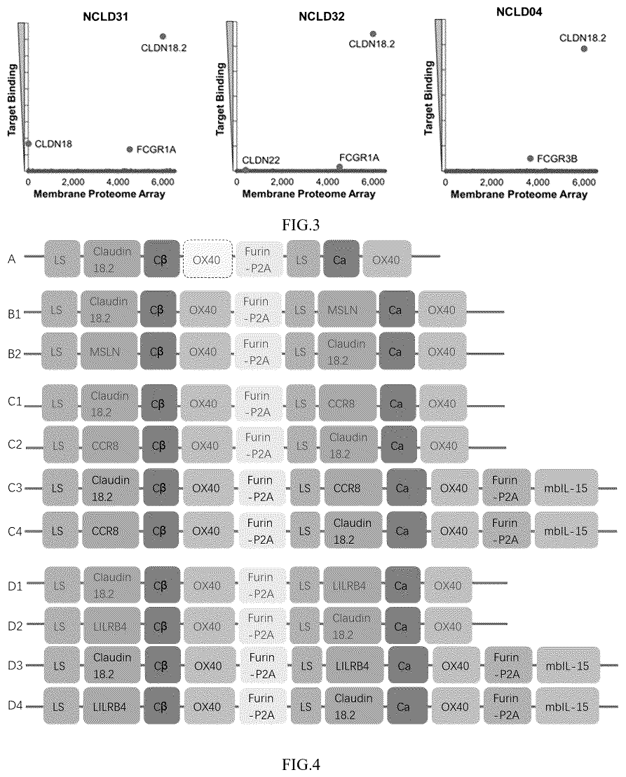

The structure of the STAR targeting Claudin18.2 is shown in FIG. 4 (A). The sequences of the Claudin18.2 nanobodies NCLD04, NCLD14, NCLD31 and NCLD32 were respectively linked to the β constant region of the STAR molecule via (G3S)4 (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is directly linked to the OX40 co-stimulatory domain), and then inserted into a lentiviral vector using the homologous recombination method to construct a complete Claudin18.2 STAR plasmid.

Lentix-293T cells were seeded into 10 cm culture dishes at a density of 5×10⁵ cells/mL and cultured in an incubator at 37° C. with 5% CO₂. When the cell density reached approximately 80% (observed under a microscope), perform transfection. Four plasmids (PMD2.G:PRSV-Rev:PMDIg:transfer plasmid=1:1:2:4) were mixed evenly with 500 μL of serum-free DMEM. 54 μL of PEI-max was mixed evenly with 500 μl of serum-free DMEM and left to stand at room temperature for 5 min (the volume-mass ratio of PEI-Max to the plasmid is 3:1). The PEI-max mixture was slowly added to the plasmid mixture, gently pipetted to mix well, and then left to stand at room temperature for 15 min. The final mixture was slowly added to the culture medium, mixed thoroughly, and then placed back in the incubator for further culture for 12-16 hours. Then, the cells were cultured in 6% FBS DMEM medium, and the virus-containing supernatant was collected at 48 hours and 72 hours.

Jurkat-C4 cells with knocked out TCR were inoculated into flat-bottomed 96-well plates at a density of 1.5×10⁵ cells/mL, and 100 μL of 1640 medium containing 10% FBS and 0.2 μL of 1000×polybrene was added to each well. The virus was serially diluted 10-fold with complete 1640 medium. The diluted cells were added to the virus wells at 100 μL/well, mixed at 32° C., centrifuged at 1500 rpm for 90 min, and then cultured in an incubator at 37° C. with 5% CO₂. After 72 hours, the infection efficiency was measured by flow cytometry. When calculating the titer, wells with an infection rate of 2-30% were selected, and the calculation formula is: Titer (TU/mL)=1.5×10⁴×positive rate÷virus volume (μL)×1000. The above-mentioned virus was used to infect T cells to express STAR.

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus-containing supernatant was added, and the plates were centrifuged at 1500 rpm for 90 min and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

For the detection of STAR infection efficiency, the above vectors were used to package lentiviruses, which were then used to infect T cells. After one-week of culture, the cells were stained with anti-mTCRβ antibody, and the membrane expression level of STAR on the cells was detected by flow cytometry. The results demonstrated that all STAR-conjugated antibodies NCLD04, NCLD14, NCLD31, and NCLD32 exhibited favorable membrane loading efficiencies, with NCLD04-STAR achieving a membrane localization rate of 80.1%.

### 3.2 In Vitro Killing Screening of Claudin18.2 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells. Experimental Objective: To validate the killing efficacy of Claudin18.2 antibody-based STAR-T cells against tumor cells expressing the Claudin18.2 target.

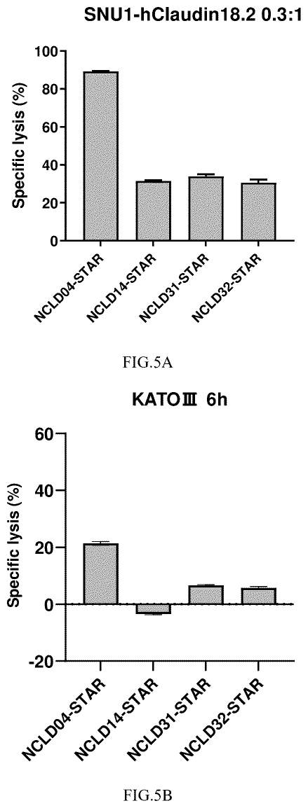

Two types of target cells, SNU1-hClaudin18.2 and KATOIII, were constructed respectively to verify the killing efficacy of NCLDs-STAR-T cells against tumor cells expressing the Claudin18.2 target. The vectors of NCLD04-STAR, NCLD14-STAR, NCLD31-STAR and NCLD32-STAR were expressed in T cells. The two types of target cells, SNU1-hClaudin18.2 and KATOIII, were seeded in 24-well plates at a density of 4×10⁵ cells per well respectively. According to different ratios of STAR-positive T cells to target cells, the corresponding numbers of NCLDs-STAR-T cells were added to the target cells for co-culture, with a co-culture volume of 1 mL. For the well plate of the target cells SNU1-hClaudin18.2, the co-cultured cell suspension was collected for detection after 24 hours of co-culture. For the well plate of the target cells KATOIII, the suspension was collected for detection after 6 hours of co-culture. Then, a luciferase reporter gene detection kit was used to detect the LUC luminescence value, and the killing efficiency of STAR-T cells against the target cells was calculated. As shown in FIGS. 5A-5B, In vitro killing effect against SNU1-hClaudin18.2 target cells: The killing efficiency of NCLD04-STAR against target cell SNU1-hClaudin18.2 is relatively strong, while the STAR flow cytometry binding and killing efficiency of NCLD14, NCLD31, and NCLD32 are relatively poor.

In vitro killing effect against KATOIII target cells: NCLD04-STAR displayed relatively high killing effect against the target cells KATOIII. In contrast, NCLD31-STAR and NCLD32-STAR showed relatively weaker killing effect against the target cells, and NCLD14-STAR exhibited no cytotoxicity against the target cells.

### 3.3 In Vitro Killing Screening of Claudin18.1 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells. Experimental Objective: To validate the killing efficacy of Claudin18.2 antibody-based STAR-T cells against tumor cells expressing the Claudin18.1 target.

In the experiment, the NCLDs-STAR vector was expressed in T cells. The SNU1-hClaudin18.1 target cells were seeded into 24-well plates at a density of 4×10⁵ cells/well. According to the ratios of STAR-positive T cells to target cells of 0.3:1, the corresponding numbers of STAR-T cells were added to the target cells for co-culture, with a co-culture volume of 1 ml. After 24 hours of co-culture, the co-culture cell suspension was collected. A luciferase reporter gene detection kit was used to detect the LUC luminescence value, and the killing efficiency of STAR-T cells against the target cells was calculated.

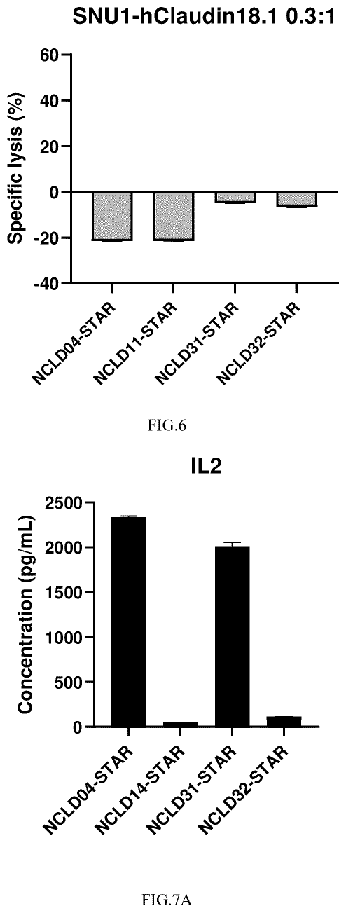

As shown in FIG. 6, in the killing experiment of SNU1-hClaudin18.1 target cells, The STARs of the antibodies NCLD04, NCLD14, NCLD31 and NCLD32 did not have specific killing effects on the target cells expressing Claudin18.1.

Therefore, based on the above killing experiments using both SNU1-hClaudin18.1 and SNU1-hClaudin18.2 target cells, the antibodies NCLD04, NCLD14, NCLD31 and NCLD32 were identified as exhibiting specific killing activity against SNU1-hClaudin18.2 target cells.

### 3.4 Cytokine Secretion of Claudin18.2 STAR

Based on the aforementioned killing experiments, T cells with relatively high killing efficiency were screened out from NCLDs-STAR-T. After the above experiments, the supernatants were collected after co-culturing the above-mentioned T cells and target cells. The secretion levels of IFN-γ, IL-2, and TNF-α were detected by ELISA.

During the activation of T cells, a large number of cytokines are released to assist T cells kill target cells or promote their expansion. Common cytokines include TNF-α, IFN-γ, and IL-2. After T cells are stimulated by target cells or antigens, the T cells are collected, centrifuged, and the supernatant is taken. The ELISA kits used for TNF-α, IFN-γ, and IL-2 are Human IL-2 Uncoated ELISA, Human TNF-α Uncoated ELISA, and Human IFN-γ Uncoated ELISA (with product numbers 88-7025, 88-7346, and 88-7316 respectively). The specific steps are as follows: Dilute the 10× Coating Buffer to 1× with ddH₂O, add the coating antibody (250×), mix well, and then add to a 96-well plate (specially for ELISA) at a volume of 100 μL per well.

Seal it with plastic wrap and leave it overnight at 4° C. Wash it 3 times with 1×PBST (also known as Wash Buffer, which is 1× PBS added with 0.05% Tween 20), 260 μL per well each time. Dilute the 5× ELISA/ELISPOT Diluent to 1× with ddH₂O, add to the 96-well plate at a volume of 200 μL per well, and let it stand at room temperature for 1 hour.

Wash it once with PBST. Dilute the standard curve (the ranges are 2-250, 4-500, and 4-500 respectively). Dilute the samples 20-50 times with 1×Diluent. Add the samples and the standard curve, 100 μL per well, with two duplicate wells. Incubate at room temperature for 2 hours, then wash 3 times with PBST. Add the Detection antibody diluted with 1×Diluent, incubate for 1 hour, and then wash 3 times with PBST. Then add HRP diluted with 1×Diluent, incubate for 30 minutes, wash 6 times. Add TMB for color development, ensuring the color development time does not exceed 15 minutes. Add 2N H₂SO₄ to terminate the reaction. And detect the absorbance at 450 nm.

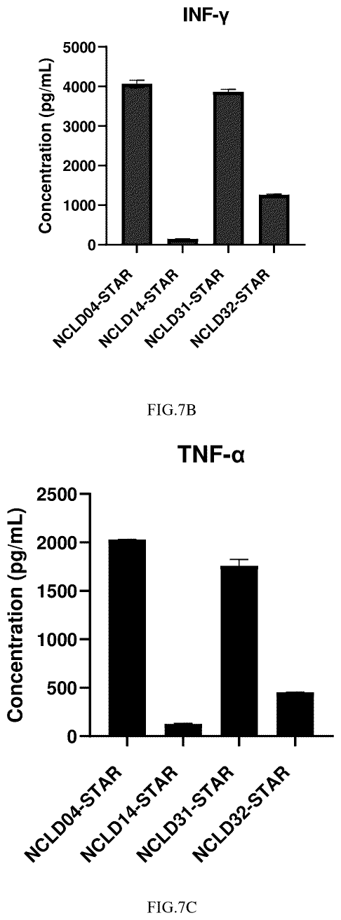

As shown in FIGS. 7A-7C, co-culturing T cells with target cells SNU1-hClaudin18.2 significantly stimulated the secretion of IL-2, TNF-α, and IFNγ by T cells. Among the antibodies, NCLD04-STAR and NCLD31-STAR exhibited the highest cytokine concentrations, demonstrating superior tumor-killing effects compared to other antibody sequences. In contrast, NCLD14-STAR and NCLD32-STAR showed relatively lower cytokine concentrations and weaker tumor-killing efficacy. 3.5. In vivo functional validation of Claudin18.2 STAR In order to further verify the in vivo inhibitory effect of Claudin18.2 STAR-T (antibodies NCLD04, NCLD14, NCLD31, NCLD32) cells in a mouse transplantation model of Claudin18.2-positive tumor cells.

The target cells of SNU1-Claudin18.2 (1:1 Matrigel) were constructed. The target cells were subcutaneously inoculated into 6- to 8-week-old female NPG mice at a dose of 2×10\hat{ }6 cells per mouse. On the 6th day after infusion, the above-mentioned T cells were selected. antibodies NCLD04, NCLD14, NCLD31, NCLD32 STAR-T cells and T cells without STAR infection (MOCK-T) were infused via the tail vein at a dose group of 4×10\hat{ }6 cells per mouse. One day before the reinfusion, and on the 5th, 9th, 12th, 15th, 22nd, 26th, 30th, and 34th days after the reinfusion, the tumor growth was detected by the luciferin substrate-catalyzed luminescence method, and the tumor fluorescence value, body weight changes were also detected.

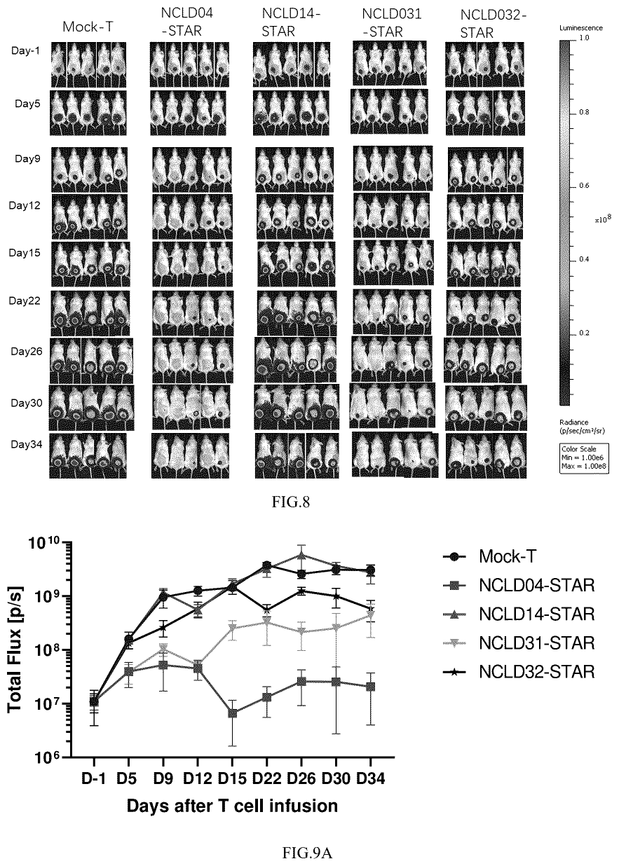

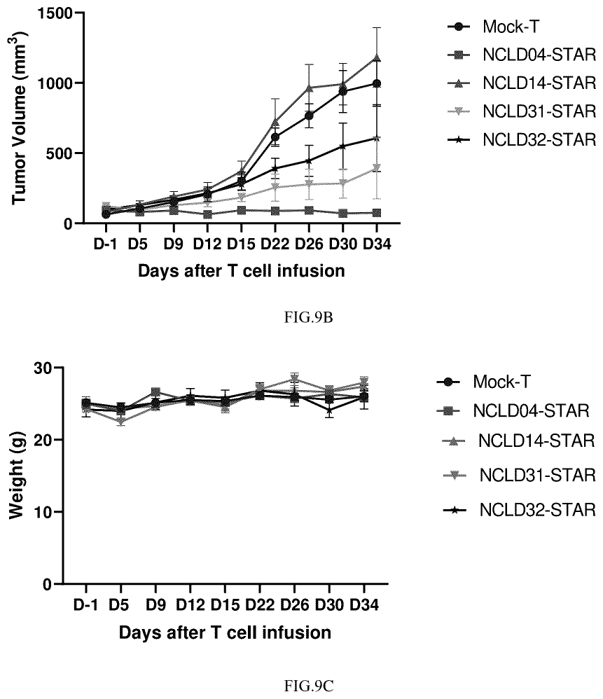

As shown in FIG. 8 and FIGS. 9A-9C, in vivo killing effect and potential safety in the lung and gastric tumor models demonstrated that NCLDs-STAR-T cells have good tumor inhibitory effects on SNU1-Claudin18.2 target cells. Among them, NCLD04-STAR-T has the strongest in vivo anti-tumor efficacy, with no abnormalities in body weight or pathological analysis observed in any group, indicating no significant toxicity. The order of the anti-tumor effects in vivo of other STAR-T cells (from strong to weak) is NCLD31-STAR-T, NCLD32-STAR-T, and NCLD14-STAR-T successively. There are also no abnormalities found in the body weight and pathological analysis of the mice in each group, and no obvious toxicity is observed.

### Example 4. Dual-target MSLN-Claudin18.2 STAR-T

### 4.1. Nanobody and STAR Targeting MSLN

The inventors previously obtained a nanobody, NM5 (SEQ ID NO:34), specifically targeting MSLN through a nanobody screening platform. The membrane protein array chip technology was used to prove that the NM5 antibody specifically binds to MSLN, indicating its good safety. Through BLI affinity detection and competition experiments, it was found that NM5 has a high affinity for MSLN (KD=1.14E-08M) and has a unique epitope recognized near the membrane. Based on this, NM5 STAR was constructed. The constant region of the α chain of NM5 STAR is based on TRAC-Cys-TM, the constant region of the β chain is based on TRBC-Cys-TM, and the constant regions of both the α chain and the β chain are directly connected to the cytoplasmic region of OX40. The NM5 nanobody is fused to the N-terminus of the constant region of the β chain. The amino acid sequence of the α chain of NM5 STAR is shown in SEQ ID NO:40; the amino acid sequence of the β chain is shown in SEQ ID NO:39. The α chain and β chain of NM5 STAR also contain a GM-CSF signal peptide (SEQ ID NO:38) at the N-terminus, which will be excised in the cells after expression.

Compared with other nanobodies, T cells expressing NM5 STAR have a stronger anti-tumor effect both in vitro and in vivo. Based on this research work, the MSLN STAR-T was modified to improve the persistence of STAR-T cells, thereby enhancing the anti-tumor effect in vivo.

### 4.2. Vector Construction and Virus Packaging

The coding nucleic acid sequences of the structures shown in FIG. 4 (B1-B2) and Table 4 were commercially synthesized and assemble, and then inserted into a lentiviral vector using the homologous recombination method to construct a complete MSLN-Claudin18.2 STAR plasmid. For the specific method, refer to Example 3.1.

Lentix-293T cells were inoculated into a 10 cm culture dish at a density of 5×10⁵ cells/mL. When the cell density reached about 80%, transfection was carried out. The ratio of the four plasmids was PMD2.G:PRSV-Rev:PMDIg:transfer plasmid=1:1:2:4, and the volume-to-mass ratio of PEI-Max to the plasmid was 3:1. After 12 hours to 16 hours the medium was replaced, and the viral supernatants were collected at 48 hours and 72 hours respectively.

The virus was serially diluted 10-fold in a 96-well plate. Then, Jurkat-C5 cells with the TCR knocked out were added to the virus wells at a density of 1.5×10⁵ cells/mL. The plate was centrifuged at 32° C., 1500 rpm for 90 minutes, followed by incubation in a culture incubator. After 72 hours, the infection efficiency was measured by a flow cytometer. The wells with an infection rate of 2% to 30% were selected for titer calculation. The titer (TU/mL)=1.5×10⁴×positive rate÷virus volume (μL)×1000.

TABLE 4
STAR Structure and Corresponding Peptide Chain Sequences

| | | STAR Peptide Chains |
|---|---|---|
| Abbreviation | STAR-T Structure | SEQ ID NO |
| NM5-STAR | NM5β-STAR-abOX40 | 39, 40 |
| NCLD04-STAR | NCLD04β- | 49, 50 |
| | a(G4S)3OX40(gh) | |
| NCLD14-STAR | NCLD14β- | 51, 52 |
| | a(G4S)3OX40(gh) | |
| NCLD04β-NM5α- | NCLD04β-NM5α-STAR- | 61, 62 |
| STAR | abOX40 | |
| NM5β-NCLD04α- | NM5β-NCLD04α-STAR- | 59, 60 |
| STAR | abOX40 | |

### 4.3 In Vitro Killing Screening of Dual-Target MSLN-Claudin18.2 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells. Construct Claudin18.2 single-positive target cells (SNU1-hClaudin18.2 target cells), MSLN single-positive target cells (SKOV3 target cells), and Claudin18.2 and MSLN double-positive target cells (KATOIII target cells) to validate the tumor-killing efficacy of MSLN-Claudin18.2 STAR-T cells against tumor cells. The above-mentioned target cells were seeded into 24-well plates at a density of 4×10⁵ cells/well. The above-mentioned STAR vector was expressed in T cells. According to the ratios of positive T cells to target cells of 0.3:1 and 1:1, the corresponding numbers of STAR-T cells were added to the target cells for co-culture, with a co-culture volume of 1 ml. After 24 hours of co-culture, the co-culture cell suspension was taken, and the LUC luminescence value was detected using a luciferase reporter gene detection kit to calculate the killing efficiency of STAR-T cells against the target cells.

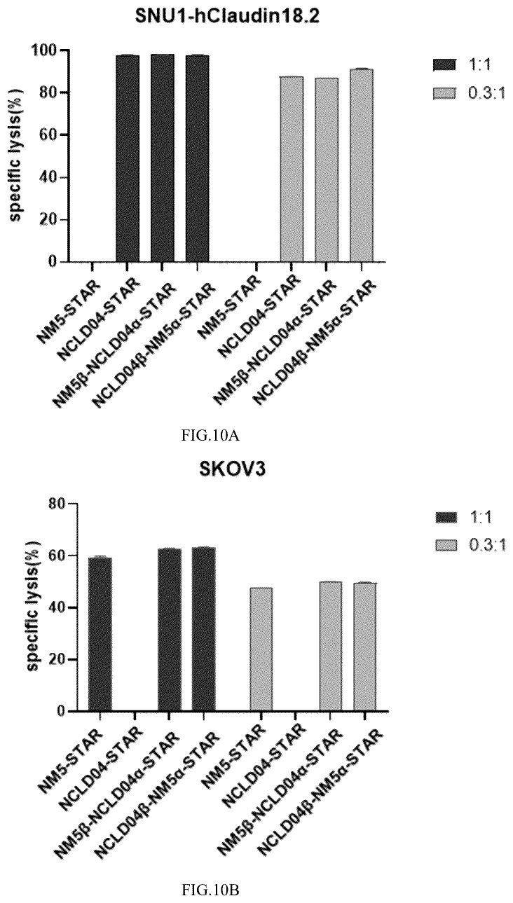

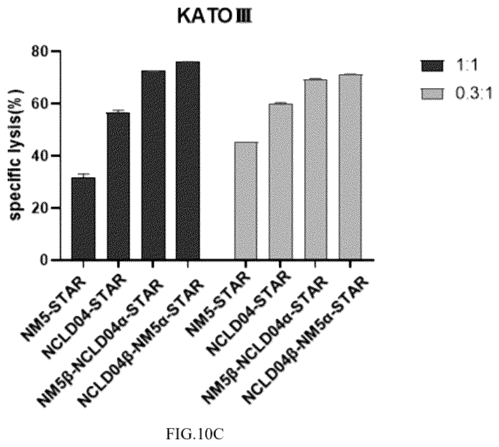

As shown in FIG. 10A, for the Claudin18.2 single-positive target cells, namely SNU1-hClaudin18.2 target cells, T cells expressing NCLD04-STAR, NM50-NCLD04α-STAR, and NCLD04β-NM5α-STAR all showed obvious killing efficiency against the target cells. For the MSLN single-positive target cells, namely SKOV3 target cells (As shown in FIG. 10B), T cells expressing NM5-STAR, NM50-NCLD04α-STAR, and NCLD04β-NM5α-STAR all showed obvious killing efficiency against the target cells. For the Claudin18.2 and MSLN double-positive target cells, namely KATOIII target cells (As shown in FIG. 10C), the NCLD04β-NM5α-STAR T cells had the best killing efficiency, followed by NM50-NCLD04α-STAR T cells, and then the single-target NCLD04-STAR and NM5-STAR T cells.

### 4.4. In Vivo Functional Validation of Dual-Target MSLN-Claudin18.2 STAR

In order to further verify in vivo inhibitory effect of Claudin18.2 STAR-T cells in the mouse xenograft model of Claudin18.2-positive tumor cells, SNU1 target cells (a mixture of SNU1-hClaudin18.2, SNU1-hMSLN and SNU1-hClaudin18.2-hMSLN at a ratio of 1:1:1) were constructed. The target cells were subcutaneously inoculated into 6-8-week-old female NPG mice at a dose of 2×10⁶ cells per mouse. On the 6th day after infusion, NM5-STAR, NCLD04-STAR, NCLD04β-NM5α-STAR T cells, and non-infected STAR T cells (MOCK-T) were infused via the tail vein at dose groups of 3×10⁶ cells per mouse and 6×10⁶ cells per mouse. Then, on days-1, 4, 7, 11, 19, and 27 (36, 42, 49, 56), the tumor growth was detected by the luciferin substrate-catalyzed luminescence method, the tumor fluorescence value and body weight change were also detected.

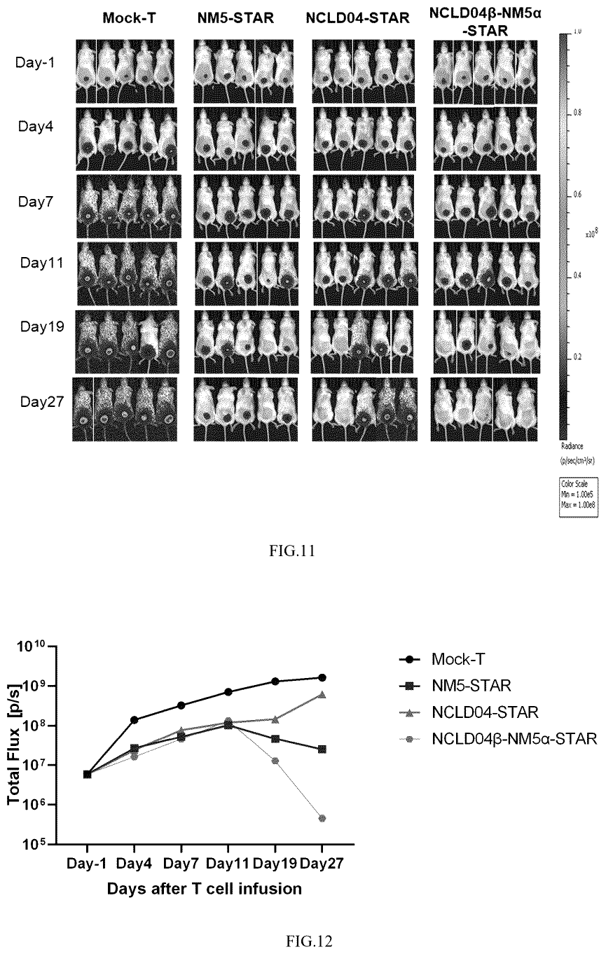

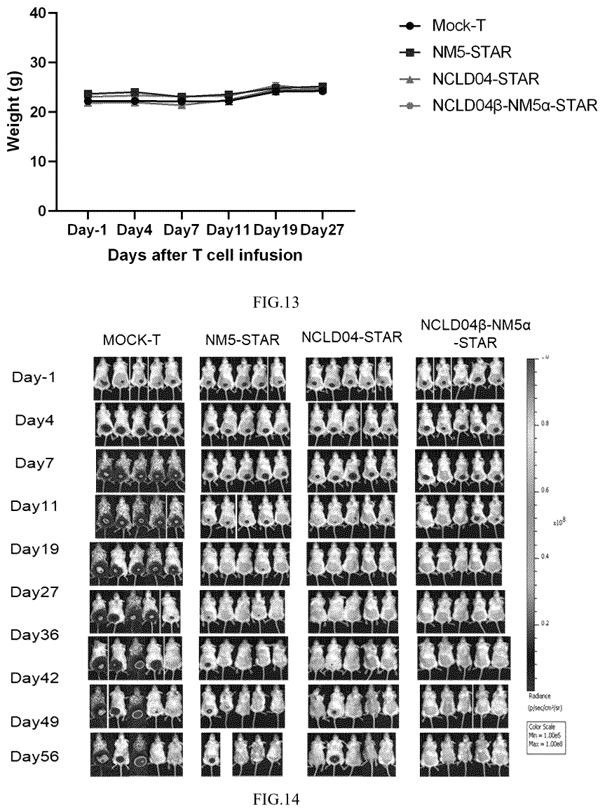

The above-mentioned STAR-T cells all showed good tumor inhibitory effects on the target cells which were a mixture of SNU1-hClaudin18.2, SNU1-hMSLN and SNU1-hClaudin18.2-hMSLN at a ratio of 1:1:1. When STAR-T cells were administered at a dose of 3E6, the therapeutic efficacy of NCLD04β-NM5α-STAR was superior to that of NM5-STAR and NCLD04-STAR. In terms of the survival analysis, NCLD04β-NM5α-STAR, NM5-STAR and NCLD04-STAR showed relatively stable survival rates within the first 27 days (as shown in FIGS. 11-13).

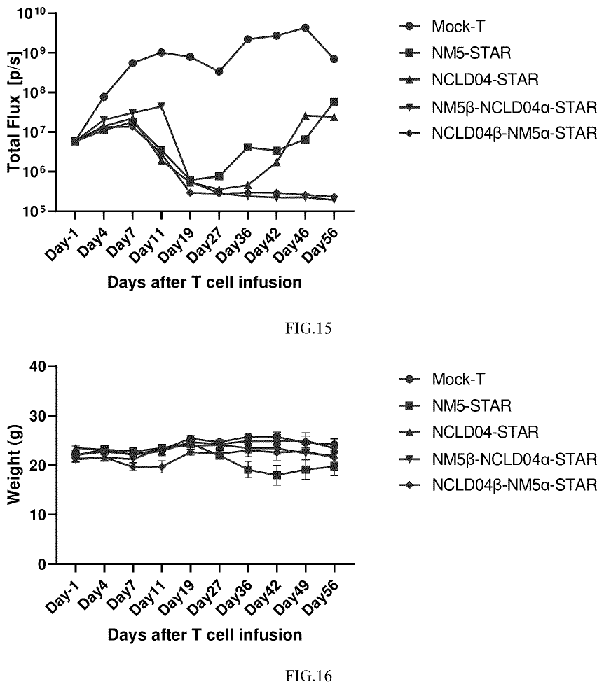

When STAR-T cells were administered at a dose of 6E6, the therapeutic efficacy of NCLD04β-NM5α-STAR was also superior to that of NM5-STAR and NCLD04-STAR. Additionally, no significant weight loss was observed in the mice treated with these T cells, indicating a favorable safety profile. In terms of survival analysis, NCLD04β-NM5α-STAR and NCLD04-STAR showed relatively stable survival rates within the first 56 days (see FIGS. 14-16).

### Example 5: Dual-Target CCR8-Claudin18.2 STAR with Co-Expressed mbIL-15

### 5.1 Nanobody and STAR Targeting CCR8

The inventor obtained a nanobody, NCR802 (SEQ ID NO:44), specifically targeting CCR8 through a nanobody screening platform. It was further validated that this antibody could be used to construct CCR8-targeting STAR-T cells, which effectively kill CCR8+target cells. Similar to Example 3, dual-target CCR8-Claudin18.2 STAR-T (FIG. 4 (C1 and C2)) was constructed, which demonstrated stronger anti-tumor effects both in vitro and in vivo. Based on the dual-target CCR8-Claudin18.2 STAR, mbIL-15 was connected via furin-P2A (FIG. 4 (C3 and C4)) to construct an mbIL-15 co-expressing CCR8-Claudin18.2 STAR.

### 5.2 Vector Construction and Virus Packaging

Referring to Example 3, the Claudin18.2-CCR8-STAR structure targeting both Claudin18.2 and CCR8 was constructed as shown in FIG. 4 (C1-C4) and Table 5. The sequences of the Claudin18.2 nanobody NCLD04 and the CCR8 antibody NCR802 were co-assembled with the constant regions of the STAR molecule (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is linked directly to the OX40 co-stimulatory domain) respectively, and were inserted into a lentiviral vector using the homologous recombination method to construct a complete Claudin18.2-CCR8-STAR plasmid.

Referring to Example 3, a Claudin18.2-CCR8-STAR structure targeting both Claudin18.2 and CCR8 was constructed. On the basis of this STAR, mbIL-15 was connected via furin-P2A (the vector structure is shown in FIG. 4). The nucleic acid sequences encoding the corresponding proteins were synthesized and assembled, and then inserted into a lentiviral vector using the homologous recombination method to construct the STAR-T plasmid.

Lentix-293T cells were seeded into 10 cm culture dishes at a density of 5×10⁵ cells/mL and cultured in an incubator at 37° C. with 5% CO₂. When the cell density reached approximately 80% (observed under a microscope), perform transfection. Four plasmids (PMD2.G:PRSV-Rev:PMDIg:transfer plasmid=1:1:2:4) were mixed evenly with 500 μL of serum-free DMEM. 54 μL of PEI-max was mixed evenly with 500 μl of serum-free DMEM and left to stand at room temperature for 5 min (the volume-mass ratio of PEI-Max to the plasmid is 3:1). The PEI-max mixture was slowly added to the plasmid mixture, gently pipetted to mix well, and then left to stand at room temperature for 15 min. The final mixture was slowly added to the culture medium, mixed thoroughly, and then placed back in the incubator for further culture for 12-16 hours. Then, the cells were cultured in 6% FBS DMEM medium, and the virus-containing supernatant was collected at 48 hours and 72 hours.

Jurkat-C4 cells with knocked out TCR were inoculated into flat-bottomed 96-well plates at a density of 1.5×10⁵ cells/mL, and 100 μL of 1640 medium containing 10% FBS and 0.2 μL of 1000×polybrene was added to each well. The virus was serially diluted 10-fold with complete 1640 medium. The diluted cells were added to the virus wells at 100 μL/well, mixed at 32° C., centrifuged at 1500 rpm for 90 min, and then cultured in an incubator at 37° C. with 5% CO₂. After 72 hours, the infection efficiency was measured by flow cytometry. When calculating the titer, wells with an infection rate of 2%-30% were selected, and the calculation formula is: Titer (TU/mL)=1.5×10⁴×positive rate÷virus volume (μL)×1000. The above-mentioned virus was used to infect T cells to express STAR.

TABLE 5
STAR Structure and Corresponding Peptide Chain Sequences

| | | STAR Peptide Chains |
|---|---|---|
| Abbreviation | STAR-T Structure | SEQ ID NO |
| NCLD04-STAR | NCLD04β-STAR- | 49, 50 |
| | a(G4S)3OX40(gh) | |
| NCR802-STAR | NCR802β-STAR- | 53, 54 |
| | abOX40 | |
| NCLD04β-NCR802α- | NCLD04β-NCR802α- | 55, 56 |
| STAR | STAR-abOX40 | |
| NCR802β-NCLD04α- | NCR802β-NCLD04α- | 57, 58 |
| STAR | STAR-abOX40 | |
| MND-NCLD04-NCR802 | MND-NCLD04β- | 55, 56(co-expressed |
| STAR-mL15 | NCR802α-STAR- | with mbIL-15) |
| | abOX40-mbIL-15 | |
| MND-NCR802-NCLD04 | MND-NCR802β- | 57, 58(co-expressed |
| STAR-mL15 | NCLD04α-STAR- | with mbIL-15) |
| | abOX40-mbIL-15 | |

5.3 Co-Expression Detection of Dual-Target and mbIL-15 Co-Expressed CCR8-Claudin18.2 STAR

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus solution was added, and the plates were centrifuged at 1500 rpm for 90 minutes and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

The above-mentioned vectors were used to package lentiviruses and infect T cells. After one week of culture, the expression levels of STAR and Claudin18.2 on the cells were detected by flow cytometry.

Specifically, the expression of the Claudin18.2 antibody in the STAR structure on the cell membrane was detected by staining with the Claudin18.2 Protein antibody, and the positive rate of STAR vector infection was detected by mTCRβ. Detection of STAR Positive Rate: The protein binding of NCLD04β-NCR802α-STAR and NCR802-STAR is the best, reaching over 65%. The protein binding of NCR8020-NCLD04α-STAR and NCLD04 STAR is relatively good, exceeding 55%. Both MND-NCLD04β-NCR802a STAR-mL15 and MND-NCR8020-NCLD04α STAR-mL15 can be successfully expressed on the membrane.

5.4 In Vitro Killing Screening of Dual-Target and mbIL-15 Co-Expressed CCR8-Claudin18.2 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells. A 293T-Claudin18.2 target cell line, a single-positive target cell line expressing Claudin18.2, was constructed to verify the killing level of CCR8-Claudin18.2 STAR-T cells against tumor cells expressing Claudin18.2. The 293T-Claudin18.2 target cells that only express the Claudin18.2 target were seeded into 24-well plates at a density of 4×10⁵ cells/well. The above-mentioned STAR vectors were expressed in T cells. According to the ratios of positive T cells to target cells of 3:1 and 1:1, the corresponding numbers of STAR-T cells were added to the target cells for co-culture, with a co-culture volume of 1 ml. After 24 hours of co-culture, the co-culture cell suspension was taken, and the LUC luminescence value was detected using a luciferase reporter gene detection kit to calculate the killing efficiency of STAR-T cells against the target cells.

As shown in FIG. 17, in the system of 293T-Claudin18.2 target cells (Claudin18.2 single-positive target cells), NCLD04-STAR, NCLD04β-NCR802α-STAR, NCR8020-NCLD04α-STAR, NCLD04β-NCR802α-STAR-mbIL-15, and NCR8020-NCLD04α-STAR-mbIL-15 all had good killing efficiency against the target cells, and the STAR-T cells co-expressing mbIL-15 had a stronger killing effect than dual-target or single-target STARs.

CCR8 single-positive target cell lines, 293T-hCCR8, were constructed to verify the killing level of CCR8-Claudin18.2 STAR-T cells against tumor cells expressing CCR8. The 293T-Claudin18.2 target cells were seeded into 24-well plates at a density of 4×10⁵ cells/well. The above-mentioned STAR vectors were expressed in T cells. The above-mentioned STAR-T cells were co-cultured with the target cells at ratios of 3:1 and 1:1, with a total co-culture volume was 1 mL. After 24 hours of co-culture, the co-culture cell suspension was taken, and the LUC luminescence value was detected using a luciferase reporter gene detection kit to calculate the killing efficiency of STAR-T cells against the target cells.

As shown in FIG. 18, in the 293T-hCCR8 cell line (CCR8 single-positive target cells), NCR802-STAR, NCLD04β-NCR802α-STAR, NCR8020-NCLD04α-STAR, NCLD04β-NCR802α-STAR-mbIL-15, and NCR8020-NCLD04α-STAR-mbIL-15 all had good killing efficiency against the target cells, and the STAR-T cells co-expressing mbIL-15 had a stronger killing effect than dual-target or single-target STARs.

5.5 Cytokine Secretion Detection of Dual-Target and mbIL-15 Co-Expressed CCR8-Claudin18.2 STAR

After the above experiments, the supernatants were collected after co-culturing the above-mentioned T cells and target cells. The secretion levels of IFN-γ, IL-2, and TNF-α were detected by ELISA. According to the experimental operation described in Section 3.4 (Cytokine Secretion of Claudin18.2 STAR) of Example 3, the secreted factors from 293T-Claudin18.2 target cells and 293T-hCCR target cells co-cultured with STAR-T cells were analyzed respectively.

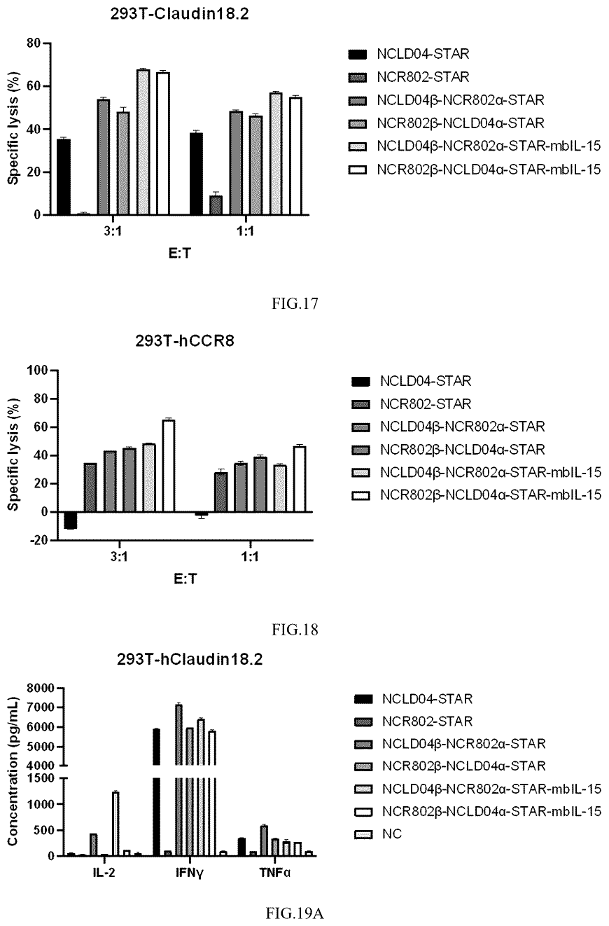

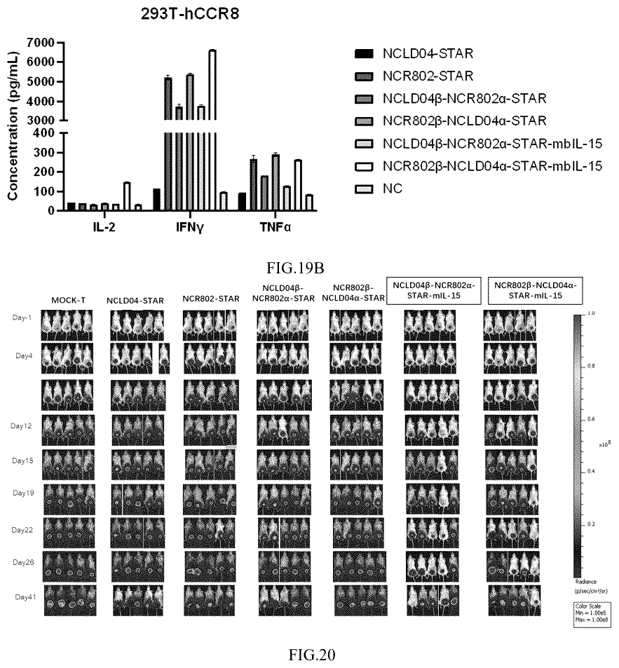

As shown in FIGS. 19A-19B, for 293T-hClaudin18.2 target cells, the factor secretion of NCLD04β-NCR802α-STAR was significantly higher than that of NCR8020-NCLD04α-STAR. Moreover, the combination of NCLD04β-NCR802α-STAR and mbIL-15 could significantly increase the secretion of IL-2. Similarly, for 293T-hCCR8 target cells, the factor secretion of NCR8020-NCLD04α-STAR was significantly higher than that of NCLD04β-NCR802α-STAR, and the combination of NCR8020-NCLD04α-STAR and mbIL-15 could significantly increase the secretion of IL-2.

5.6 In Vivo Functional Validation of Dual-Target and mbIL-15 Co-Expressed CCR8-Claudin18.2 STAR

To further verify the in-vivo inhibitory effect of CCR8-Claudin18.2 STAR-T cells in a mouse transplantation model of Claudin18.2-positive tumor cells, SNU1-Claudin18.2 fluorescently labeled target cells were constructed. These target cells were subcutaneously inoculated into 6-8-week-old female NPG mice at a dose of 2×10⁶ cells/mouse. On the 8th day after inoculation, NCLD04-STAR, NCR802-STAR, NCLD04β-NCR802α-STAR, NCR8020-NCLD04α-STAR, NCLD04β-NCR802α-STAR-mbIL-15, and NCR802β-NCLD04α-STAR-mbIL-15 were infused via the tail vein at a dose of 3×10⁶ cells/mouse. Then, on days-1, 4, 7, 14, 21, 27, 34 and 42, the tumor growth was detected by the luciferin substrate-catalyzed luminescence method, and the tumor fluorescence value, and body weight change were also detected.

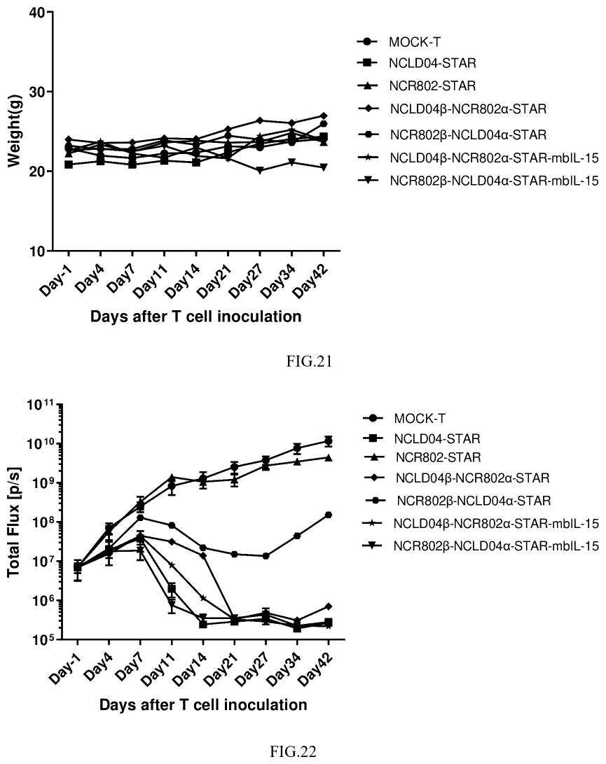

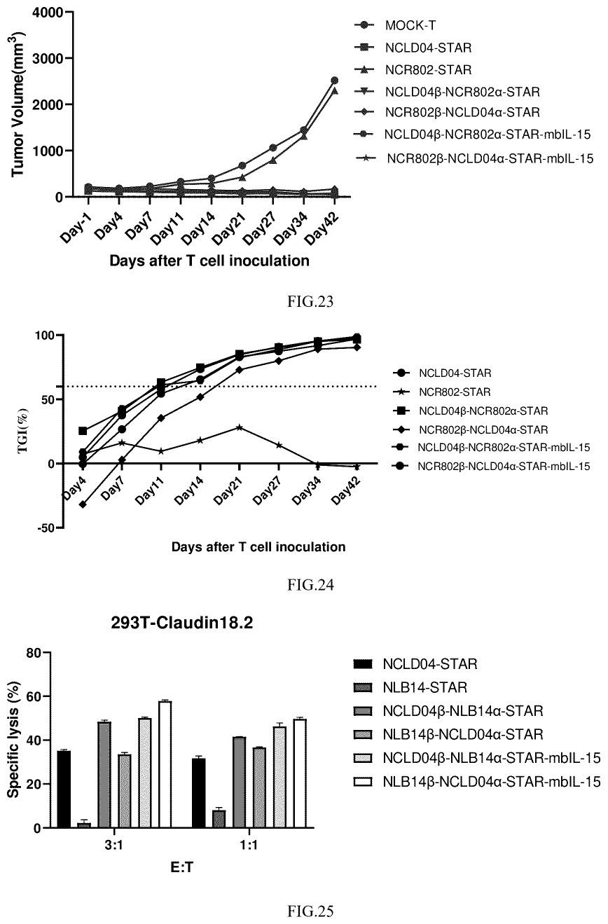

In the test of in-vivo killing effect and potential safety issues in the gastric cancer model, as shown in FIGS. 20-24, NCLD04-STAR, NCLD04β-NCR802α-STAR, NCR8020-NCLD04α-STAR, NCLD04β-NCR802α-STAR-mbIL-15, and NCR802β-NCLD04α-STAR-mbIL-15 all showed good tumor-inhibitory effects on SNU1-Claudin18.2 target cells. Among them, the NCR8020-NCLD04α-STAR-mbIL-15, NCLD04-STAR, and NCLD04β-NCR802α-STAR-mbIL-15 cells showed the best tumor-inhibitory effects on the target cells, the NCLD04β-NCR802α-STAR and NCR8020-NCLD04α-STAR cells had a moderate tumor-inhibitory effect on the target cells, while NCR802-STAR had the weakest tumor-inhibitory effect on the target cells. Meanwhile, the body weights of the mice treated with these several types of T cells did not show significant decreases, indicating relatively good safety. The STAR-T cells with the structures of NCLD04β-NCR802α-mbIL-15 and NCR8020-NCLD04α-STAR-mbIL-15 had the strongest in-vivo expansion ability, could maintain a higher proportion of CD8-positive T cells, but caused higher toxic reactions in mice.

### Example 6. Dual-target LILRB4-Claudin18.2 STAR with Co-expressing mbIL-15

6.1. Nanobody and STAR Targeting LILRB4 Previously, aiming at the LILRB4 target, the inventor obtained a nanobody NBL14 (SEQ ID NO:48) specifically targeting LILBR4 through a nanobody screening platform. Similar to Example 3, dual-target LILRB4-Claudin18.2 STAR-T (FIG. 4 (D1 and D2)) was constructed, which demonstrated stronger anti-tumor effects both in vitro and in vivo. Based on the dual-target LILRB4-Claudin18.2 STAR, mbIL-15 was connected via furin-P2A (FIG. 4 (D3 and D4)) to construct an mbIL-15 co-expressing LILRB4-Claudin18.2 STAR.

### 6.2. Vector Construction and Virus Packaging

Referring to Example 3, the Claudin18.2-LILRB4-STAR structure targeting both Claudin18.2 and LILRB4 was constructed as shown in FIG. 4 and Table 6. The sequences of the Claudin18.2 nanobody NCLD04 and the LILRB4 antibody NLB14 were co-assembled with the constant regions of the STAR molecule (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is linked directly to the OX40 co-stimulatory domain) respectively, and were inserted into a lentiviral vector using the homologous recombination method to construct a complete Claudin18.2-LILRB4-STAR plasmid.

Referring to Example 3, a Claudin18.2-LILRB4-STAR structure targeting both Claudin18.2 and LILRB4 was constructed, and mbIL-15 was connected to this STAR via furin-P2A (the vector structure is shown in FIG. 4). The corresponding nucleic acid sequences were synthesized and assembled, and then inserted into the lentiviral vector using the homologous recombination method to construct the STAR-T plasmid.

Lentix-293T cells were inoculated into a 10 cm culture dish at a density of 5×10⁵ cells/mL. When the cell density reached about 80%, transfection was carried out. The ratio of the four plasmids was PMD2.G:PRSV-Rev:PMDIg:transfer plasmid=1:1:2:4, and the volume-to-mass ratio of PEI-Max to the plasmid was 3:1. After 12 hours to 16 hours the medium was replaced, and the viral supernatants were collected at 48 hours and 72 hours respectively.

The virus was serially diluted 10-fold in a 96-well plate. Then, Jurkat-C5 cells with the TCR knocked out were added to the virus wells at a density of 1.5×10⁵ cells/mL. The plate was centrifuged at 32° C., 1500 rpm for 90 minutes, followed by incubation in a culture incubator. After 72 hours, the infection efficiency was measured by a flow cytometer. The wells with an infection rate of 2% to 30% were selected for titer calculation. The titer (TU/mL)=1.5×10⁴×positive rate÷virus volume (μL)×1000.

TABLE 6
STAR Structure and Corresponding Peptide Chain Sequences

| | | STAR Peptide Chains |
|---|---|---|
| Abbreviation | STAR-T Structure | SEQ ID NO |
| NCLD04β-STAR | NCLD04β-STAR- | 49, 50 |
| | a(G4S)3OX40(gh) | |
| NLB14β-STAR | NLB14β-STAR- | 63, 64 |
| | a(G4S)3OX40(gh) | |
| NCLD04-NLB14-STAR | NCLD04β-NLB14α- | 65, 66 |
| | STAR-abOX40 | |
| NLB14-NCLD04-STAR | NLB14β-NCLD04α- | 67, 68 |
| | STAR-abOX40 | |
| NCLD04-NLB14-STAR- | MND-NCLD04β- | 65, 66(co-expressed |
| mbIL-15 | NLB14α-STAR- | with mbIL-15) |
| | abOX40-mbIL-15 | |
| NLB14-NCLD04-STAR- | MND-NLB14β- | 67, 68(co-expressed |
| mbIL-15 | NCLD04α-STAR- | with mbIL-15) |
| | abOX40-mbIL-15 | |

6.3. Co-Expression Detection of Dual-Target and mbIL-15 Co-Expressed LILRB4-Claudin18.2 STAR

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus solution was added, and the plates were centrifuged at 1500 rpm for 90 minutes and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

The above-mentioned vectors were used to package lentiviruses and infect T cells. After one week of culture, the expression level of STAR on the cell membrane was detected by flow cytometry using anti mTCRβ antibody staining. The results showed that the positive rate of STAR detection was: NCLD04β-STAR, NLB140-STAR, NCLD04β-NLB14α-STAR, NLB140-NCLD04α-STAR, NCLD04β-NLB14α-STAR-mbIL-15, and NLB14β-NCLD04α-STAR-mbIL-15 all can be successfully expressed on the membrane.

6.4 In Vitro Killing of Dual-Target and mbIL-15 Co-Expressed LILRB4-Claudin18.2 STAR

The Claudin18.2 single-positive target cells, 293T-Claudin18.2 and KATOIII, were constructed. The target cells were seeded in 24-well plates at a density of 4×10⁵ cells per well respectively. According to the ratios of positive T cells to target cells of 3:1 and 1:1, STAR-T cells were added to the 293T-Claudin18.2 target cells for co-culture, with a co-culture volume of 1 ml. According to the ratios of positive T cells to target cells of 1:1 and 0.3:1, STAR-T cells were added to the KATOIII target cells for co-culture, with a co-culture volume of 1 ml. After 24 hours of co-culture the two types of target cells, the co-culture cell suspension was taken, and the LUC luminescence value was detected using a luciferase reporter gene detection kit to calculate the killing efficiency of STAR-T cells against the target cells.

As shown in FIG. 25, for the Claudin18.2 single-positive target cells 293T-Claudin18.2 and KATOIII, NCLD04-STAR, NCLD04β-NLB14α-STAR, NLB140-NCLD04α-STAR, NCLD04β-NLB14α-STAR-mbIL-15 and NLB14β-NCLD04α-STAR-mbIL-15 all had good killing efficiency against the target cells. Moreover, in the 293T-Claudin18.2 cells, the STAR-T cells co-expressing mbIL-15 had enhanced killing effects compared with the dual-target or single-target STAR.

The LILRB4 single-positive target cells, 293T-hLILRB4 and MV4-11, was constructed. Target cells were seeded in 24-well plates at a density of 4×10⁵ cells per well respectively. According to the ratios of positive T cells to target cells of 3:1 and 1:1, STAR-T cells were added to the 293T-hLILRB4 target cells for co-culture, with a co-culture volume of 1 ml. According to the ratios of positive T cells to target cells of 1:1 and 0.3:1, STAR-T cells were added to the MV4-11 target cells for co-culture, with a co-culture volume of 1 ml. After 24 hours of co-culture the two types of target cells, take the co-cultured cell suspension, detect the LUC luminescence value using a luciferase reporter gene detection kit, and calculate the killing efficiency of the STAR-T cells against the target cells.

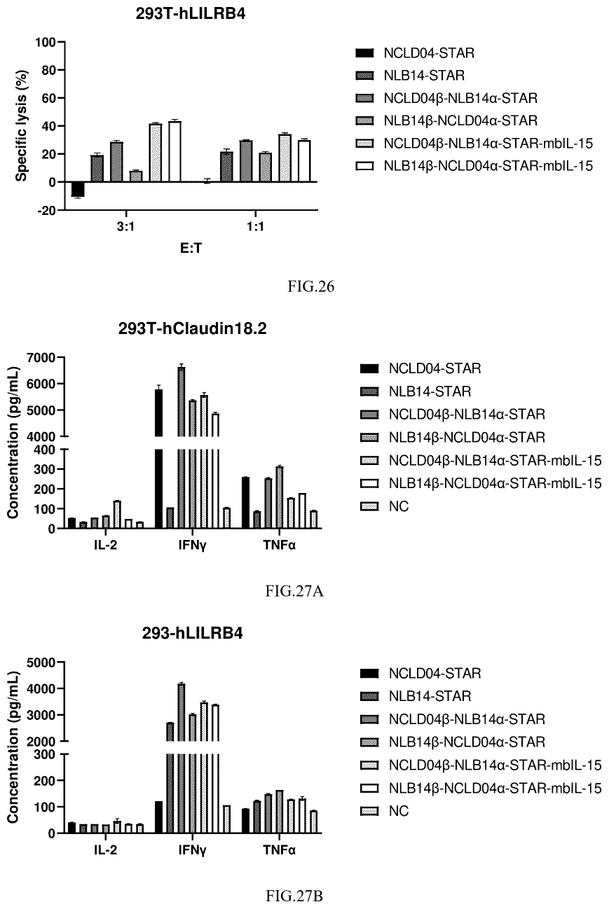

As shown in FIG. 26, for the 293T-hLILRB4 target cells, the in vitro killing efficiency of STAR-T cells co-expressing mbIL-15 is higher than that of single-target or dual-target STAR-T cells. For the MV4-11 target cells, all the STAR-T cells exhibit good in vitro killing efficiency.

6.5 Cytokine Secretion Detection of Dual-target and mbIL-15 Co-expressed LILRB4-Claudin18.2 STAR-T cell

After the above experiments, the supernatants were collected after co-culturing the above-mentioned T cells and target cells. The secretion levels of IFN-γ, IL-2, and TNF-α were detected by ELISA. Following the experimental procedures in Section 3.4 (Cytokine Secretion of Claudin18.2 STAR) of Example 3, the cytokine secretion profiles were analyzed for 293T-Claudin18.2 target cells and 293T-hLILRB4 target cells after co-culture with STAR-T cells.

As shown in FIGS. 27A-27B, consistent with the conclusion of Claudin18.2-CCR8-STAR, NCLD04β-NLB14α-STAR demonstrated significantly enhanced cytokine secretion compared to NLB140-NCLD04α-STAR when co-cultured with 293T-hClaudin18.2 target cells. Moreover, the combination of NCLD04β-NLB14α-STAR with mbIL-15 notably increased IL-2 secretion. However, no corresponding cytokine secretion trends were observed among the groups when co-cultured with 293T-hLILRB4 target cells.

### Example 7: Claudin18.2-CAR-T and Dual-Target CCR8-Claudin18.2 CAR-T

### 7.1 Construction of Claudin18.2 CAR and Dual-Target CCR8-Claudin18.2 CAR Vectors and Virus Packaging

The VHH sequence of the Claudin18.2-positive antibody NCLD04 obtained from the above-mentioned sequencing was assembled with other modules of the CAR molecule (including but not limited to the hinge region, transmembrane region, co-stimulatory domain, signal-transduction domain, VHH region of the antibody targeting the tumor antigen, and/or linker sequences) to construct CARs with different structures, which were then inserted into a lentiviral vector using the homologous recombination method to construct Claudin18.2 CAR plasmids.

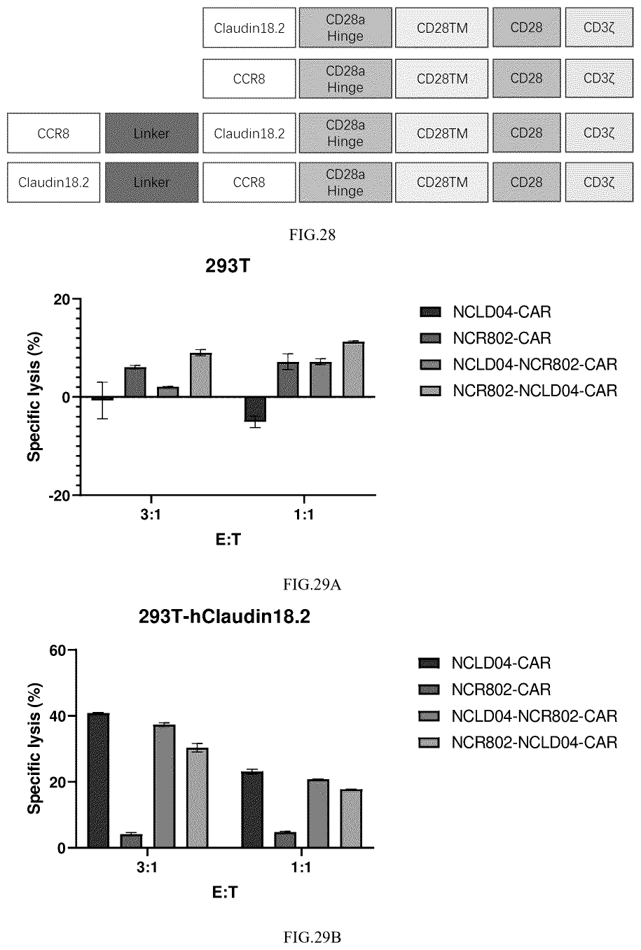

The CAR structure targeting both CCR8 and Claudin18.2 is shown in FIG. 28 and Table 7. The Claudin18.2 nanobody NCLD04 and the CCR8 antibody NCR802 were respectively connected through linkers such as (G4S)₄, (G4S)₃, or (EAAAK)₃(SEQ ID NO:25), and then jointly assembled onto the CAR molecular structure. They were inserted into a lentiviral vector using the homologous recombination method to construct a complete CCR8-Claudin18.2-CAR plasmid.

TABLE 7
CAR structures and corresponding peptide chain sequences

| CAR structures | SEQ ID NO |
|---|---|
| NCLD04-CAR | 69 |
| NCLD14-CAR | 70 |
| NCR802-CAR | 71 |
| NCR802-(G4S)4-NCLD04-CAR | 72 |
| NCLD04-(G4S)4-NCR802-CAR | 73 |

### 7.2 Detection of Infection Efficiency of CCR8-Claudin18.2 CAR-T Cells

After obtaining PBMCs by the Ficoil separation method, the cells were counted. And then 1.5 times the amount of CD3/CD28 Dynabeads was added and incubated for 45 minutes. The cells were then cultured at a density of 1.2×10⁶ cells/mL. After 24 hours, the cells were infected with the virus at an MOI of 2. Following another 24 hours, the medium was replaced, and the cells were passaged every other day thereafter. At 72 hours post-infection, the infection efficiency of the CAR was analyzed by detecting the proportion of fluorescently labeled (RFP) cells and the binding of VHH antibodies using flow cytometry. All single-target and dual-target CAR-T structures could successfully bind to the cell membrane and had a good binding ability to the protein.

### 7.3 In Vitro Killing Efficiency Detection of CCR8-Claudin18.2 CAR-T Cells

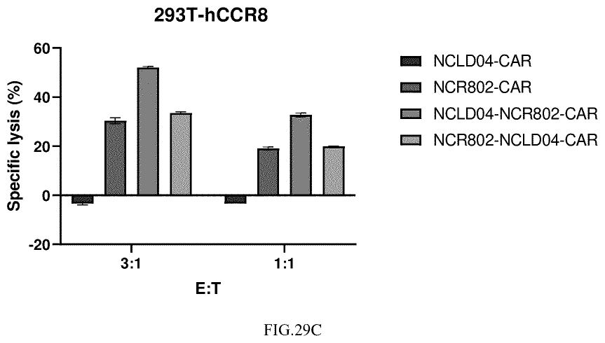

293T cells, 293T target cells overexpressing hCCR8, and 293T target cells overexpressing hClaudin18.2 were constructed. The above three types of target cells were seeded into 24-well plates at a density of 1.5×10⁵ cells/well. After 24 hours, according to the ratios of CAR-positive T cells to target cells of 3:1 and 1:1, the corresponding numbers of CAR-T cells were added to the target cells. After 24 hours of culture, the killing efficiency of CAR-T cells against the target cells was detected (as shown in FIGS. 29A-29C).

For the 293T target cells, NCLD04-CAR-T had no killing effect on the target cells, and NCR802-CAR-T, NCR802-NCLD04-CAR-T, and NCLD04-NCR802-CAR-T had weak killing efficiencies against the target cells.

For the 293T target cells overexpressing hClaudin18.2, both the single-target NCLD04-CAR-T and dual-target CAR-T cells had killing efficiency. The killing efficiency of CAR-T cells against the target cells was ranked as: NCLD04-CAR-T>NCLD04-NCR802-CAR-T>NCR802-NCLD04-CAR-T, wherein NCR802-CAR-T had no killing effect.

For the 293T target cells overexpressing hCCR8 both the single-target NCR802-CAR-T and dual-target CAR-T cells had killing efficiency. The killing efficiency of CAR-T cells against the target cells was ranked as: NCLD04-NCR802-CAR-T>NCR802-NCLD04-CAR-T and NCR802-CAR-T; wherein NCLD04-CAR-T had no killing effect.

The above-described are only the preferred embodiments of the present invention and do not impose any formal limitations on the present invention. Although the specific implementation manners of the present invention have been described above, those skilled in the art should understand that this is only for illustrative purposes. The protection scope of the present invention is defined by the appended claims. Those skilled in the art can make various changes or modifications to the disclosed technical content without departing from the scope of the technical solution of the present invention, and these changes and modifications all fall within the protection scope of the present invention.

What is claimed:

1. A single-domain antibody that specifically binds to Claudin18.2, comprising CDR1, CDR2 and CDR3 sequences selected from CDR1, CDR2 and CDR3 in SEQ ID NO:4 or 8.

2. The single-domain antibody according to claim 1, which comprises CDR1, CDR2 and CDR3 selected from any of the following groups:
(1) CDR1 as shown in SEQ ID NO: 1, CDR2 as shown in SEQ ID NO: 2, and CDR3 as shown in SEQ ID NO: 3;
(2) CDR1 as shown in SEQ ID NO: 5, CDR2 as shown in SEQ ID NO: 6, and CDR3 as shown in SEQ ID NO: 7.

3. The single-domain antibody according to claim 1, wherein the amino acid sequence has at least 80%, 90%, 95%, or 99% identity to SEQ ID NO: 4 or 8, or, wherein the amino acid sequence shown in SEQ ID NO: 4 or 8.

4. A synthetic T-cell receptor and antigen receptor (STAR) targeting Claudin18.2, wherein the target-binding region of the STAR comprises the single-domain antibody specifically binding to Claudian18.2 according to claim 1.

5. The STAR according to claim 4, wherein the STAR comprises a first peptide chain and a second peptide chain:
i) the first peptide chain comprises a first target-binding region and a first constant region, and the second peptide chain comprises a second target-binding region and a second constant region; or,
ii) the first peptide chain comprises a first constant region, the second peptide chain comprises a second constant region, and the first peptide chain or the second peptide chain comprises a first target-binding region;
wherein the first target-binding region and/or the second target-binding region comprises one or more antigen-binding regions, and the multiple antigen-binding regions are the same or different;
the antigen-binding region in the first target-binding region and/or the second target-binding region comprises the Claudin18.2 single-domain antibody according to claim 1.

6. The STAR according to claim 4, wherein the STAR comprises any one of the following groups:
a) the first peptide chain comprises a first constant region; the second peptide chain sequentially comprises, from the N-terminus to the C-terminus, at least one Claudin18.2 single-domain antibody according to claim 1, and a second constant region;
b) the first peptide chain comprises, from the N-terminus to the C-terminus, the Claudin18.2 single-domain antibody according to claim 1, and a first constant region; the second peptide chain sequentially comprises, from the N-terminus to the C-terminus, an antibody or antigen-binding fragment specifically binding to LILRB4, MSLN or CCR8, and a second constant region;
c) the first peptide chain comprises a first constant region; the second peptide chain comprises, from the N-terminus to the C-terminus, at least one Claudin18.2 single-domain antibody according to claim 1, an antibody or an antigen-binding fragment specifically binding to CCR8, MSLN or LILRB4, and a second constant region;
In the each of the above group a)-c), the first constant region of the first peptide chain is a TCRα chain constant region or a TCRβ chain constant region, and the second constant region of the second peptide chain is a TCRβ chain constant region or a TCRα chain constant region; the constant regions of the first peptide chain and the second peptide chain are not simultaneously TCRα chain constant regions, nor are they simultaneously TCRβ chain constant regions.

7. The STAR according to claim 6, wherein the first constant region is a TCRα chain constant region or a TCRβ chain constant region or a modified TCRα chain constant region or TCRβ chain constant region;
wherein the second constant region is a TCRα chain constant region or a TCRβ chain constant region or a modified TCRα chain constant region or TCRβ chain constant region.

8. The STAR according to claim 7, wherein the TCRα chain constant region comprises an amino acid sequence shown in one of SEQ ID NOs:9-15, and/or the TCRβ chain constant region comprises an amino acid sequence shown in one of SEQ ID NOs:16-22.

9. The STAR according to claim 6, wherein the first peptide chain and/or the second peptide chain has at least one exogenous intracellular functional domain linked to its C-terminus;
wherein the exogenous intracellular functional domain is an endodomain of OX40, the endodomain of OX40 comprises the amino acid sequence of SEQ ID NO:23;
wherein the exogenous intracellular functional domain is linked directly or via a linker to the C-terminus of the constant region of the first peptide chain and/or the second peptide chain.

10. The STAR according to claim 6, wherein the STAR is co-expressed with a membrane-bound IL-15 protein (mbIL-15); wherein:
i) the amino acid sequence of IL-15 is shown in SEQ ID NO: 27;
ii) the amino acid sequence of the extracellular domain of IL-15Rα is shown in SEQ ID NO: 28;
iii) the amino acid sequence of the linker connecting the extracellular domain of IL-15Rα to IL-15 is shown in SEQ ID NO: 24-25, 29; and/or
iv) the amino acid sequence of mbIL-15 is shown in SEQ ID NO: 30.

11. The STAR according to claim 4, wherein:
a) the STAR comprises a first peptide chain shown in SEQ ID NO:49 and a second peptide chain shown in SEQ ID NO:50
b) the STAR comprises a first peptide chain shown in SEQ ID NO:51 and a second peptide chain shown in SEQ ID NO:52;
c) the STAR comprises a first peptide chain shown in SEQ ID NO:61 and a second peptide chain shown in SEQ ID NO:62;
d) the STAR comprises a first peptide chain shown in SEQ ID NO:59 and a second peptide chain shown in SEQ ID NO:60;
e) the STAR comprises a first peptide chain shown in SEQ ID NO:55 and a second peptide chain shown in SEQ ID NO:56;
f) the STAR comprises a first peptide chain shown in SEQ ID NO:57 and a second peptide chain shown in SEQ ID NO:58;
g) the STAR comprises a first peptide chain shown in SEQ ID NO:65 and a second peptide chain shown in SEQ ID NO:66; or
h) the STAR comprises a first peptide chain shown in SEQ ID NO:67 and a second peptide chain shown in SEQ ID NO:68.

12. A chimeric antigen receptor (CAR) targeting Claudin18.2, comprising an extracellular antigen-binding region, wherein the extracellular antigen-binding region comprises the Claudin18.2 single-domain antibody according to claim 1, and the CAR sequentially comprises, from the N-terminus to the C-terminus, an extracellular antigen-binding region, a hinge region, a transmembrane domain, a co-stimulatory domain, and an intracellular signal-transduction domain.

13. The CAR according to claim 12, wherein the extracellular antigen-binding region further comprises an antigen-binding region specifically binding to another antigen;
wherein the other antigen is CCR8, the antigen-binding region specifically binding to CCR8 comprises CDR1 as shown in SEQ ID NO:41, CDR2 as shown in SEQ ID NO:42, and CDR3 as shown in SEQ ID NO:43;
wherein the other antigen is MSLN, the antigen-binding region specifically binding to MSLN comprises CDR1 as shown in SEQ ID NO:31, CDR2 as shown in SEQ ID NO:32, and CDR3 as shown in SEQ ID NO:33;
or, wherein the other antigen is LILRB4, the antigen-binding region specifically binding to LILRB4 comprises CDR1 as shown in SEQ ID NO:45, CDR2 as shown in SEQ ID NO:46, and CDR3 as shown in SEQ ID NO:47.

14. The CAR according to claim 12, wherein the CAR comprises an amino acid sequence shown in any one of SEQ ID NOs:69-73.

15. An isolated therapeutic immune cell comprising a STAR or a CAR;
wherein a target-binding region of the STAR or an extracellular antigen-binding region of the CAR comprises the single-domain antibody specifically binding to Claudin18.2 according to claim 1.

16. The therapeutic immune cell according to claim 15, wherein the immune cell is a T cell or natural killer (NK) cell.

17. A method for preparing the therapeutic immune cell according to claim 15, comprising:
step 1) providing a starting immune cell;
step 2) introducing an expression vector into the starting immune cell; and
step 3) harvesting the immune cell obtained in step 2);
Wherein the expression vector comprises an nucleic acid molecule encoding the single-domain antibody, a STAR, or a CAR;
Wherein the single-domain antibody comprises CDR1, CDR2 and CDR3 sequences selected from CDR1, CDR2 and CDR3 in SEQ ID NO:4 or 8;
Wherein a target-binding region of the STAR comprises the single-domain antibody;
Wherein an extracellular antigen-binding region of the CAR comprises the single-domain antibody.

18. A pharmaceutical composition comprising a single-domain antibody, a STAR, a CAR, and/or, a therapeutic immune cell, and a pharmaceutically acceptable carrier; Wherein the single-domain antibody comprises CDR1, CDR2 and CDR3 sequences selected from CDR1, CDR2 and CDR3 in SEQ ID NO:4 or 8;
Wherein a target-binding region of the STAR comprises the single-domain antibody;
Wherein an extracellular antigen-binding region of the CAR comprises the single-domain antibody;
Wherein the therapeutic immune cell comprises the STAR or the CAR.

19. A method for treating a disease in a subject, comprising administering to the subject a therapeutically effective amount of a single-domain antibody, a STAR, a CAR, a therapeutic immune cell, and/or a pharmaceutical composition;
Wherein the single-domain antibody comprises CDR1, CDR2 and CDR3 sequences selected from CDR1, CDR2 and CDR3 in SEQ ID NO:4 or 8;
Wherein a target-binding region of the STAR comprises the single-domain antibody;
Wherein an extracellular antigen-binding region of the CAR comprises the single-domain antibody;
Wherein the therapeutic immune cell comprises the STAR or the CAR;
Wherein the pharmaceutical composition comprises the single-domain antibody, the STAR or the CAR;
wherein the disease is a Claudin18.2-related disease, wherein the Claudin18.2-related disease is a Claudin18.2-related autoimmune disease, hematologic tumor, or solid tumor.

20. The method according to claim 19,
wherein the autoimmune disease is selected from: systemic lupus erythematosus (SLE), polymyositis and dermatomyositis, systemic scleroderma, Sjögren's syndrome, autoimmune hemolytic anemia, or rheumatoid arthritis;
wherein the hematologic tumor or solid tumor is selected from: lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, digestive tract cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, mesothelioma, B-cell malignancies or sarcoma.
